SEQUENCE LISTING (55 sequences)

SEQ ID NO: 1 - DNA, 21 bp, Artificial sequence
  Synthetic construct
  misc_feature (1)..(21): residues 1-2, 2-3, 16-17, 17-18, 18-19, 19-20 and 20-21 are connected by thio ester bonds
ggggacgacgtcgtggggggg

SEQ ID NO: 2 - DNA, 20 bp, Artificial sequence
  Synthetic construct
  misc_feature (1)..(20): residues 1-2, 2-3, 15-16, 16-17, 17-18, 18-19 and 19-20 are connected by thio ester bonds
gggggagcatgctggggggg

SEQ ID NO: 3 - protein, 9 aa, Artificial sequence
  Synthetic construct
CDCRGDCFC

SEQ ID NO: 4 - protein, 8 aa, Artificial sequence
  Synthetic construct
GGCDGRCG

SEQ ID NO: 5 - protein, 9 aa, Artificial sequence
  Synthetic construct
CRRETAWAC

SEQ ID NO: 6 - protein, 9 aa, Artificial sequence
  Synthetic construct
CDCRGDCFC

SEQ ID NO: 7 - protein, 6 aa, Artificial sequence
  Synthetic construct
  misc_feature (5)..(5): Xaa can be any naturally occurring amino acid
RGDWXE

SEQ ID NO: 8 - protein, 6 aa, Artificial sequence
  Synthetic construct
TRGDTF

SEQ ID NO: 9 - protein, 7 aa, Artificial sequence
  Synthetic construct
  misc_feature (5)..(6): Xaa can be any naturally occurring amino acid
RGDLXXL

SEQ ID NO: 10 - protein, 7 aa, Artificial sequence
  Synthetic construct
  misc_feature (1)..(2): Xaa can be any naturally occurring amino acid
  misc_feature (5)..(6): Xaa can be any naturally occurring amino acid
XXDLXXL

SEQ ID NO: 11 - protein, 5 aa, Artificial sequence
  Synthetic construct
SRGDM

SEQ ID NO: 12 - protein, 9 aa, Artificial sequence
  Synthetic construct
VVISYSMPD

SEQ ID NO: 13 - protein, 7 aa, Artificial sequence
  Synthetic construct
IELLQAR

SEQ ID NO: 14 - protein, 21 aa, Artificial sequence
  Synthetic construct
CNGRCGGKLAKLAKKLAKLAK

SEQ ID NO: 15 - protein, 9 aa, Artificial sequence
  Synthetic construct
CVSNPRWKC

SEQ ID NO: 16 - protein, 9 aa, Artificial sequence
  Synthetic construct
CHVLWSTRC

SEQ ID NO: 17 - protein, 8 aa, Artificial sequence
  Synthetic construct
CWDDGWLC

SEQ ID NO: 18 - protein, 9 aa, Artificial sequence
  Synthetic construct
CPCFLLGCC

SEQ ID NO: 19 - protein, 8 aa, Artificial sequence
  Synthetic construct
DFKLFAVY

SEQ ID NO: 20 - protein, 5 aa, Artificial sequence
  Synthetic construct
EWVDV

SEQ ID NO: 21 - protein, 4 aa, Artificial sequence
  Synthetic construct
  MISC_FEATURE (1)..(2): Xaa at residues 1 and 2 is Asp or Glu
  MISC_FEATURE (3)..(3): Xaa at residues 3 is Gly or Leu
XXXW

SEQ ID NO: 22 - protein, 10 aa, Artificial sequence
  Synthetic construct
CTTHWGFTLC

SEQ ID NO: 23 - protein, 17 aa, Artificial sequence
  Synthetic construct
  misc_feature (2)..(3): Xaa can be any naturally occurring amino acid
  misc_feature (7)..(7): Xaa can be any naturally occurring amino acid
  misc_feature (9)..(10): Xaa can be any naturally occurring amino acid
  misc_feature (12)..(16): Xaa can be any naturally occurring amino acid
NXXEIEXYXXWXXXXXY

SEQ ID NO: 24 - protein, 12 aa, Artificial sequence
  Synthetic construct
HTMYYHHYQHHL

SEQ ID NO: 25 - protein, 7 aa, Artificial sequence
  Synthetic construct
ATWLPPR

SEQ ID NO: 26 - protein, 12 aa, Artificial sequence
  Synthetic construct
WHSDMEWWYLLG

SEQ ID NO: 27 - protein, 6 aa, Artificial sequence
  Synthetic construct
RRKRRR

SEQ ID NO: 28 - protein, 10 aa, Artificial sequence
  Synthetic construct
TAASGVRSMH

SEQ ID NO: 29 - protein, 10 aa, Artificial sequence
  Synthetic construct
LTLRWVGLMS

SEQ ID NO: 30 - protein, 7 aa, Artificial sequence
  Synthetic construct
LMLPRAD

SEQ ID NO: 31 - protein, 9 aa, Artificial sequence
  Synthetic construct
CKGGRAKDC

SEQ ID NO: 32 - protein, 9 aa, Artificial sequence
  Synthetic construct
CLSSRLDAC

SEQ ID NO: 33 - protein, 8 aa, Artificial sequence
  Synthetic construct
VGLPEHTQ

SEQ ID NO: 34 - protein, 12 aa, Artificial sequence
  Synthetic construct
VPWMEPAYQRFL

SEQ ID NO: 35 - protein, 7 aa, Artificial sequence
  Synthetic construct
  misc_feature (4)..(4): Xaa can be any naturally occurring amino acid
  misc_feature (7)..(7): Xaa can be any naturally occurring amino acid
LTVXPWX

SEQ ID NO: 36 - protein, 7 aa, Artificial sequence
  Synthetic construct
  misc_feature (4)..(4): Xaa can be any naturally occurring amino acid
LTVXPWY

SEQ ID NO: 37 - protein, 12 aa, Artificial sequence
  Synthetic construct
LLGPYELWELSH

SEQ ID NO: 38 - protein, 4 aa, Artificial sequence
  Synthetic construct
RPMC

SEQ ID NO: 39 - protein, 7 aa, Artificial sequence
  Synthetic construct
YSGKWGW

SEQ ID NO: 40 - protein, 12 aa, Artificial sequence
  Synthetic construct
TSPLNIHNGQKL

SEQ ID NO: 41 - protein, 8 aa, Artificial sequence
  Synthetic construct
CGFELETC

SEQ ID NO: 42 - protein, 7 aa, Artificial sequence
  Synthetic construct
  MISC_FEATURE (7)..(7): Xaa is Gly or Ser
NSVRDLX

SEQ ID NO: 43 - protein, 7 aa, Artificial sequence
  Synthetic construct
  misc_feature (6)..(6): Xaa can be any naturally occurring amino acid
  MISC_FEATURE (7)..(7): Xaa is Ser or Ala
NSVSSXX

SEQ ID NO: 44 - protein, 9 aa, Artificial sequence
  Synthetic construct
CGNKRTRGC

SEQ ID NO: 45 - protein, 4 aa, Artificial sequence
  Synthetic construct
  misc_feature (4)..(4): Xaa can be any naturally occurring amino acid
EGRX

SEQ ID NO: 46 - protein, 4 aa, Artificial sequence
  Synthetic construct
  misc_feature (1)..(1): Xaa can be any naturally occurring amino acid
  MISC_FEATURE (4)..(4): Xaa is Gly or Val
XFGX

SEQ ID NO: 47 - protein, 10 aa, Artificial sequence
  Synthetic construct
CVSSNPRWKC

SEQ ID NO: 48 - protein, 9 aa, Artificial sequence
  Synthetic construct
CHVLWSTRC

SEQ ID NO: 49 - protein, 9 aa, Artificial sequence
  Synthetic construct
SWCEPGWCR

SEQ ID NO: 50 - protein, 8 aa, Artificial sequence
  Synthetic construct
DPRATPGS

SEQ ID NO: 51 - protein, 7 aa, Artificial sequence
  Synthetic construct
SMSIARL

SEQ ID NO: 52 - protein, 9 aa, Artificial sequence
  Synthetic construct
CGRRAGGSC

SEQ ID NO: 53 - protein, 9 aa, Artificial sequence
  Synthetic construct
CSCFRDVCC

SEQ ID NO: 54 - protein, 7 aa, Artificial sequence
  Synthetic construct
TPKTSVT

SEQ ID NO: 55 - protein, 7 aa, Artificial sequence
  Synthetic construct
GLSGGRS

(19) United States
(12) Patent Application Publication
(10) Pub. No.: US 2007/0212337 A1
(43) Pub. Date: Sep. 13, 2007

(54) Polypeptide-nucleic acid conjugate for immunoprophylaxis or immunotherapy for neoplastic or infectious disorders

(75) Inventors: Atul Bedi, Timonium, MD (US); Rajani Ravi, Ruxton, MD (US); Shulin Li, Baton Rouge, LA (US)

Correspondence Address: DLA PIPER US LLP, 4365 EXECUTIVE DRIVE, SUITE 1100, SAN DIEGO, CA, 92121-2133, US

(73) Assignee: The Johns Hopkins University, Baltimore, MD (US)

(21) Appl. No.: 11/701,092

(22) Filed: Jan. 31, 2007

Related U.S. Application Data
(60) Provisional application No. 60/833,100, filed on Jul. 25, 2006.
(60) Provisional application No. 60/764,223, filed on Feb. 1, 2006.

(51) Int. Cl. A61K 48/00; A61K 39/395; C07K 16/46; C12N 5/08

(52) U.S. Cl. 424/93.21; 424/178.1; 514/44; 530/391.1; 435/455; 435/366

(57) ABSTRACT

The present invention discloses compositions which induce cross-activation of immune mediated and direct death signaling in targeted cells by exploiting the properties of a antibody/peptide-nucleic acid conjugate. The conjugate is able to simultaneously activate multiple death signaling mechanisms that are specifically targeted to neoplastic cells, including tumor cells. Methods of using the conjugate of the present invention as an immunotherapeutic modality for the treatment or prevention of neoplastic diseases or other disorders is also disclosed. Further, methods are disclosed for identifying such conjugates by assaying test agents for various cytotoxic responses, including the induction of hyperfusion between neoplastic cells in vitro.

### RELATED APPLICATIONS

This application claims priority under 35 U.S.C. §119(e) to U.S. Provisional Patent Application No. 60/833,100, filed Jul. 25, 2006, and No. 60/764,223, filed Feb. 1, 2006, each of which is hereby incorporated by reference in its entirety.

### FIELD OF THE INVENTION

The present invention relates generally to immunostimulatory therapeutic modalities and, more specifically to antibody/peptide-nucleic acid conjugates, which cross activate immune-mediated signaling and directed cell death signaling in targeted cells, and methods for the prevention or treatment of neoplastic and/or other disorders using such conjugates.

### BACKGROUND INFORMATION

Chemotherapy is a cornerstone of the current management of cancers. The induction of cell death by chemotherapeutic agents involves DNA damage-induced activation of an “intrinsic” death signaling pathway that depends on the function of the p53 tumor suppression gene. The mechanism by which p53 induces apoptosis involves transcriptional activation of proapoptotic genes such as the Bcl-2 homology 3 (BH3)-domain containing protein, PUAM (p53 upregulated modulator of apoptosis), and Noxa. These genes encode proteins which trigger mitochondrial outer membrane permeabilization via the multidomain Bcl-2 family members, BAX and BAK. The mitochondrial release of cytochrome c (cyto c) results in transactivation of caspase-9, and the liberation of Smac/DIABLO (second mitochondria-derived activator of caspase/direct IAP binding protein with low pH) facilitates the activation of effector caspases (-3 and -7) by alleviating the inhibitory effect of X-linked Inhibitor of Apoptosis Protein (XIAP). Activated caspases execute the terminal events of cell death via cleavage of critical substrates that maintain cytoskeletal and DNA integrity. Therefore, the susceptibility of tumor cells to chemotherapy-induced apoptosis is determined by a dynamic balance between p53/BAX-mediated mitochondrial death signaling and expression of survival proteins that counteract mitochondrial permeabilization (Bcl-X_{L}) and activation of effector caspases (XIAP). The vast majority of human cancers harbor genetic aberrations (loss/inactivation of death signaling proteins and/or overexpression/activation of survival signals) which reduce cellular susceptibility to apoptosis and limit the antitumor efficacy of chemotherapy. The antitumor efficacy of chemotherapeutic agents may be limited by their extrusion from cancer cells expressing mulitdrug resistance proteins, as well as dose-limiting cytotoxicity to normal tissues.

Although various approaches can be used to elicit innate and adaptive immune responses against tumor cells, the intrinsic resistance of cancer cells to immunologic cytotoxicity poses a significant limitation to the efficacy of immunotherapy. Cancer cells enhance their ability to withstand an attack by cytotoxic immune effector cells via acquisition of specific genetic alterations that interfere with the shared mitochondrial death signaling pathway entrained by Granzyme B, Interferon-γ, and Apo2 ligand/Tumor necrosis factor related apoptosis inducing ligand (Apo2L/TRAIL), three key mediators of immunologic cell-mediated cytotoxicity. Both Granzyme B and Apo2L/TRAIL transduce death signals via proteolytic activation of effector caspases-3/-7, as well as induction of mitochondrial outer membrane permeabilization via cleavage of BID to truncated forms that activate BAX and BAK. The mitochondrial release of Smac/DIABLO facilitates the activation of effector caspases-3/-7 by alleviating the inhibitory effect of XIAP. The susceptibility of tumor cells to immunologic cytotoxicity is determined by the level of expression of XIAP, as well as the ability to counter-act XIAP-mediated inhibition of effector caspases (-3/-7) via mitochondrial release of Smac. As such, cancer cells which express high levels of XIAP and also fail to trigger the mitochondrial release of Smac due to co-expression of Bcl-X_{L} may not allow activation of effector caspases (-3/-7) to a threshold required for immune cell-mediated apoptosis. Since expression/activity of BCI-X_{L}, as well as XIAP is upregulated by receptor induced signals (NF-κB, Akt, STAT3/5), the overexpression/activation of receptor signaling (e.g., epidermal growth factor receptor [EGFR], HER2/neu, insulin-like growth factor receptor-1 [IGF-1R], cytokines, co-stimulatory molecules) in cancers may limit their susceptibility to immunologic cytotoxicity. Current cancer immunotherapy may also be limited by the failure to activate and recruit immune effectors within the tumor microenvironment and/or specifically target the immune effectors to tumor cells.

### SUMMARY OF THE INVENTION

The present invention is based on a unified approach for cross-activation of immune mediated and direct death signaling in targeted cells. The present invention exploits the properties of an antibody/polypeptide-nucleic acid conjugate, which include simultaneously activating multiple death signaling mechanisms that are specifically targeted to tumor cells and overcoming the intrinsic resistance of cancer cells to standard therapeutic modalities. Further, the present invention can be used for targeted immunotherapy and immunoprophylaxis of neoplastic diseases and other disorders.

In one embodiment, an isolated antibody-nucleic acid conjugate or a peptide-nucleic acid conjugate is disclosed, including an antibody or peptide that specifically binds to a cellular component of a tumor cell, tumor vasculature, and/or a component of a tumor microenvironment, and one or more immunostimulatory nucleic acid sequences (INAS), where one or more of the nucleic acid sequences includes a pathogen-associated molecular pattern (PAMP) or other motif that can activate immune cells.

In one aspect, the antibody is a chimeric antibody, a multispecific antibody, a humanized antibody, a single chain antibody, or an Fab fragment.

In another aspect, the cellular component includes epidermal growth factor receptor (EGFR, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family; platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family; TRK receptor family; ephrin (EPH) receptor family; AXL receptor family; leukocyte tyrosine kinase (LTK) receptor family; TIE receptor family, angiopoietin 1,2; receptor tyrosine kinase-like orphan receptor (ROR) receptor family; discoidin domain receptor (DDR) family; RET receptor family; KLG receptor family; RYK receptor family; MuSK receptor family; Transforming growth factor β (TGF-β) receptors, TGF-β; Cytokine receptors, Class I (hematopoietin family) and Class II (interferon/IL-10 family) receptors, tumor necrosis factor (TNF) receptor superfamily (TNFRSF), death receptor family; cancer-testis (CT) antigens, lineage-specific antigens, differentiation antigens, alpha-actinin-4, ARTC1 , breakpoint cluster region-Abelson (Bcr-abl) fusion products, B-RAF, caspase-5 (CASP-5), caspase-8 (CASP-8), β-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: β-Dgalactose 2-α-Lfucosyltransferase (LDLR/FUT) fusion protein, HLA-A2. arginine to isoleucine exchange at residue 170 of the α-helix of the α2-domain in the HLA-A2 gene (HLA-A*201-R170I), HLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class I, NFYC, OGT, OS-9, pml-RARalpha fusion protein, PRDX5, PTPRK, K-ras (KRAS2), N-ras (NRAS), HRAS, RBAF600, SIRT2, SNRPD1, SYT-SSX1 or -SSX2 fusion protein, Triosephosphate Isomerase, BAGE, BAGE-1, BAGE-2,3,4,5, GAGE-1,2,3,4,5,6,7,8, GnT-V (aberrant N-acetyl glucosaminyl transferase V, MGAT5), HERV-K-MEL, KK-LC, KM-HN-1, LAGE, LAGE-1, CTL-recognized antigen on melanoma (CAMEL), MAGE-A1 (MAGE-1), MAGE-A2, MAGE-A3, MAGE-A4, MAGE-A5, MAGE-A6, MAGE-A8, MAGE-A9, MAGE-A10, MAGE-A11, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gp100, gp100/Pmell7 (SILV), tyrosinase (TYR), TRP-1, HAGE, NA-88, NY-ESO-1, NY-ESO-1/LAGE-2, SAGE, Sp17, SSX-1,2,3,4, TRP2-INT2, carcino-embryonic antigen (CEA), Kallikrein 4, mammaglobin-A, OA1, prostate specific antigen (PSA), TRP-1/gp75, TRP-2, adipophilin, interferon inducible protein absent in melanoma 2 (AIM-2), BING-4, CPSF, cyclin D1, epithelial cell adhesion molecule (Ep-CAM), EphA3, fibroblast growth factor-5 (FGF-5), glycoprotein 250 (gp250), EGFR (ERBB1), HER-2/neu (ERBB2), interleukin 13 receptor α2 chain (ILI 3Ralpha2), IL-6 receptor, intestinal carboxyl esterase (iCE), alpha-feto protein (AFP), M-CSF, mdm-2, MUC1, p53 (TP53), PBF, PRAME, PSMA, RAGE-1, RNF43, RU2AS, SOX10, STEAP1, survivin (BIRC5), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA661, LDHC, MORC, SGY-1, SPO11, TPX1, NY-SAR-35, FTHL17, NXF2, TDRD1, TEX15, FATE, TPTE, immunoglobulin idiotypes, Bence-Jones protein, estrogen receptors (ER), androgen receptors (AR), CD40, CD30, CD20, CD19, CD33, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), β-human chorionic gonadotropin, squamous cell carcinoma antigen, neuron-specific enolase, heat shock protein gp96, GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr virus (EBV) proteins (EBV latent membrane proteins—LMP1, LMP2; others), Hepatitis B or C virus proteins, and HIV proteins.

In another aspect, the “immunostimulatory nucleic acid sequence” (INAS) is a pathogen-associated molecular pattern (PAMP) or other motif that can activate immune cells, including, but not limited to, CpG DNA (CpG), herpes simplex virus (HSV) DNA, double stranded RNA (dsRNA), and single stranded RNA (ssRNA). Further, INAS may include one or more nucleic acid sequences that silence gene expression or induce intracellular death signaling, including but not limited to, double stranded RNA (dsRNA), short interfering RNA (siRNA), short hairpin RNA (shRNA), or micro RNA.

In a related aspect, INAS may be a coding or non-coding sequence. For example, an INAS may be SEQ ID NO:1, in one illustrative example.

In a related aspect, the conjugate includes an antibody that specifically binds to EGFR or HER2/neu and one or more immunostimulatory nucleic acid sequences, where one or more of the nucleic acid sequences includes a CpG DNA sequence as set forth in SEQ ID NO:1.

In one embodiment, an isolated peptide-nucleic acid conjugate is disclosed including a peptide that binds to a cellular component of a targeted tumor cell, tumor vasculature, and/or a component of a tumor microenvironment, and one or more INAS, where the one or more nucleic acid sequences include a pathogen-associated molecular pattern (PAMP) or other motif that can activate immune cells.

In one aspect, the peptide that is conjugated to nucleic acid sequences is derived from phage display or other sources, including αvβ1 integrin (CRRETAWAC (SEQ ID NO:5)), αvβ3 integrin (CDCRGDCFC (SEQ ID NO:6)/RGD-4C; RGDWXE (SEQ ID NO:7)), αvβ5 integrin (TRGDTF (SEQ ID NO:8)), αvβ6 (RGDLxxL (SEQ ID NO:9) or xxDLxxL (SEQ ID NO:10)), αIIβ3 (SRGDM (SEQ ID NO:11)), annexin V mimic for αvβ5 (VVISYSMPD (SEQ ID NO:12)), E-selectin (IELLQAR (SEQ ID NO:13)), Endothelial cell mitochondria (CNGRC-GG-(KLAKLAK)2 (SEQ ID NO:14)), Ephrin-A2 and Ephrin-A4 (CVSNPRWKC (SEQ ID NO:15), CHVLWSTRC (SEQ ID NO:16)), Fibronectin (CWDDGWLC (SEQ ID NO:17)), ICAM-I or von Willebrand factor (CPCFLLGCC (SEQ ID NO:18)/LLG-4C), lamin-1 (DFKLFAVY (SEQ ID NO:19)), P-selectin (EWVDV (SEQ ID NO:20)), MMP-9:integrin complex (D/E)(D/E)(G/L)W (SEQ ID NO:21), MMP-9 and MMP-2 (gelatinases) (CTTHWGFTLC (SEQ ID NO:22)), Type I cadherin on endothelium (N—Ac—CHAVC—NH2), Flt-1 region of VEGF NxxEIExYxxWxxxxxY(SEQ ID NO:23), KDR region of VEGF (HTMYYHHYQHHL (SEQ ID NO:24), ATWLPPR(SEQ ID NO:25)), VEGF receptor (WHSDMEWWYLLG (SEQ ID NO:26), RRKRRR (SEQ ID NO:27), Aminopeptidase N/CD13 (NGR), NG2 proteolgycan (TAASGVRSMH (SEQ ID NO:28), LTLRWVGLMS (SEQ ID NO:29)), Adrenal gland derived peptide (LMLPRAD (SEQ ID NO:30)), Adipose Tissue derived peptide (CKGGRAKDC SEQ ID NO:3 1)), Brain derived peptide (SR1), Brain endothelium derived peptide (CLSSRLDAC (SEQ ID NO:32)), Glioma cell derived peptide (VGLPEHTQ (SEQ ID NO:33)), Neuroblastoma derived peptide (VPWMEPAYQRFL (SEQ ID NO:34)), Bone Marrow derived peptide (GGG, GFS, LWS), Breast cancer (HER2/neu) derived peptide (LTVxPWx (SEQ ID NO:35), LTVxPWY (SEQ ID NO:36), HER2 Ab/Trastuzumab mimotope—LLGPYELWELSH (SEQ ID NO:37)), Colon derived peptide (RPMC (SEQ ID NO:38)), Intestine derived peptide (YSGKWGW (SEQ ID NO:39)), Head and Neck Squamous Cell Cancer derived peptide (TSPLNIHNGQKL (SEQ ID NO:40)), Lung vasculature derived peptide (CGFELETC(SEQ ID NO:41)), Coronary artery endothelia derived peptide (NSVRDL(G/S) (SEQ ID NO:42), NSVSSx(S/A) (SEQ ID NO:43)), Lymphatic Vessel derived peptide (CGNKRTRGC (SEQ ID NO:44)/Lyp-1), Multiple Organ derived peptide (GVL, EGRx (SEQ ID NO:45), xFG(GN) (SEQ ID NO:46)), Pancreatic Islet derived peptide (CVSSNPRWKC (SEQ ID NO:47), CHVLWSTRC (SEQ ID NO:48)), Pancreas derived peptide (SWCEPGWCR (SEQ ID NO:49)), Prostate derived peptide (AGG, DPRATPGS (SEQ ID NO:50), SMSIARL (SEQ ID NO:51), CGRRAGGSC (SEQ ID NO:52), GVL), Retina derived peptide (RDV, CSCFRDVCC (SEQ ID NO:53)), Teratogen ligand derived peptide (TPKTSVT (SEQ ID NO:54)), and Uterus derived peptide (GLSGGRS (SEQ ID NO:55)).

In another embodiment, a method of preventing or treating a neoplastic disease is disclosed including administering to a subject in need thereof, a composition including a polypeptide/peptide-nucleic acid conjugate, where the conjugate includes an polypeptide/peptide that specifically binds to a cellular component of a tumor cell, tumor vasculature, and/or a component of a tumor microenvironment and one or more immunostimulatory nucleic acid sequences, where one or more of the nucleic acid sequences include a pathogen-associated molecular pattern (PAMP).

In one aspect, the method further includes removing immune cells from the subject, contacting the cells with the conjugate ex vivo, and reintroducing the cells into the subject. In a further aspect, the method includes administering other agents including chemotherapeutic agents, ionizing radiation, hormonal therapy, cellular immunotherapy, vaccines, monoclonal antibodies, biological therapy, anti-angiogenic therapy, or small molecule-targeted therapy.

In another aspect, the neoplastic disorder includes, but is not limited to, head and neck cancers, aero-digestive cancers, gastro-intestinal cancers, esophageal cancers, stomach/gastric cancers, pancreatic cancers, hepato-biliary/liver cancers, colorectal cancers, anal cancers, small intestine cancers, genito-urinary cancers, urologic cancers, renal/kidney cancers, ureter cancers, testicular cancers, urethra/penis cancers, gynecologic cancers, ovarian/fallopian tube cancers, peritoneal cancers, uterine/endometrial cancers, cervical/vagina/vulva cancers, gestational trophoblastic disease, prostate cancers, bone cancers, sarcoma (soft tissue/bone), lung cancers, mesothelioma, mediastinum cancers, breast cancers, central nervous system cancers, brain cancers, melanoma, leukemia, lymphoma (Hodgkin's Disease and Non-Hodgkin's lymphoma), plasma cell neoplasms, myeloma, myelodysplastic syndrome, endocrine tumors, skin cancers, melanoma, thyroid cancers, parathyroid cancers, adrenal, pancreatic endocrine cancers, carcinoid, multiple endocrine neoplasia, AIDS-related malignancies, cancer of unknown primary site, and various childhood cancers. Preferably, the subject is a human.

In another embodiment, a method of identifying a nucleic acid conjugate which induces immune cell activation/maturation and target cell death is disclosed including contacting one or more cells in vitro with a test nucleic acid conjugate containing an antibody or peptide that specifically binds to a cellular component of a tumor cell, tumor vasculature, and/or a component of a tumor microenvironment, where the antibody or peptide is conjugated to a nucleic acid comprising one or more immunostimulatory nucleic acid sequences (INAS), and where one or more of the nucleic acid sequences include a pathogen-associated molecular pattern (PAMP), and determining induction of a marker or a phenotypic change in the one or more cells in the presence or absence of immune cells, where the determined induction or change in the presence of the test antibody/peptide-nucleic acid conjugate is indicative of immune cell activation/maturation, modulation of target cell signaling, and target cell death.

In another embodiment, an isolated antibody-nucleic acid conjugate is disclosed, including an antibody that binds to a cellular component of an immune cell, such as a dendritic cell (DC), and one or more immunostimulatory nucleic acid sequences (INAS), where one or more of the nucleic acid sequences includes a pathogen-associated molecular pattern (PAMP) or other motif that can activate immune cells.

In one aspect, the antibody binds to a cellular component of dendritic cells (DCs) including, but not limited to, DC antigen uptake receptors, C-type lectin-like receptors, dendritic cell-specific ICAM-3-grabbing nonintegrin (DC-SIGN, CD209), macrophage mannose receptor (MMR, MRC1), DEC-205 (LY75) and FLT3.

In one aspect, the method includes administering an antibody/peptide-nucleic acid conjugate, where the nucleic acid sequences silence gene expression or induce intracellular death signaling.

In another aspect, the antibody-nucleic acid conjugate is further conjugated with an antigen derived from tumor cells.

In another aspect, the antibody-nucleic acid conjugate is further conjugated with an antigen derived from an infectious microbe or pathogenic microorganism including viruses, bacteria, mycobacteria, spirochetes, fungi, rickettsia, mycoplasma, chiamydia, protozoan and metazoan parasites, or helminth.

In another embodiment, a method of preventing or treating a neoplastic or infectious disease is disclosed including administering to a subject in need thereof, a composition including an antibody/peptide-nucleic acid conjugate, where the conjugate includes an antibody that binds to a dendritic cell, an antigenic peptide derived from a tumor cell or microbe/pathogenic organism, and one or more immunostimulatory nucleic acid sequences (INAS), where one or more of the nucleic acid sequences includes a pathogen-associated molecular pattern (PAMP) or other motif that can activate immune cells.

Exemplary methods and compositions according to this invention are described in detail.

### BRIEF DESCRIPTION OF THE DRAWINGS

FIG. 1 illustrates nucleotide (DNA/RNA)-conjugated antibodies.

FIG. 2 illustrates nucleotide (DNA/RNA)-conjugated tumor targeted peptides.

FIG. 3 illustrates the mechanism of action of a DNA-antibody.

FIG. 4 shows immunoblots demonstrating CpG DNA-conjugated anti-EGFR antibody and anti-HER2 antibody.

FIG. 5 is an immunoblot demonstrating the inhibition of EGFR phosphorylation (Tyr 1068) by either anti-EGFR antibody (EGFR Ab) or CpG DNA-conjugated anti-EGFR antibody (EGFR Ab-CpG A/C).

FIG. 6 is a showing of flow cytometry analysis of the expansion of CD56⁺ PBMCs following treatment with EGFR antibody-CpG DNA conjugate (EGFR Ab-CpG A) but not with EGFR antibody conjugated to control DNA (control).

FIG. 7 is a showing of FACS analysis, which demonstrates the maturation of dendritic cells by CpG DNA-conjugated anti-EGFR antibody.

FIG. 8 is a graph demonstrating the induction of HT-29 tumor cell death by CpG DNA conjugated anti-EGFR antibody as a function of PBMC:tumor cell ratio.

FIGS. 9 is a graph demonstrating the induction of HT-29 tumor cell death by CpG DNA conjugated anti EGFR antibody as a function of time.

FIG. 10 shows bar graphs demonstrating the effects of CpG DNA-conjugated antibodies on the expression of Interferon-γ (IFN-γ) and Apo2L/TRAIL in PBMCs. A) shows the quantification of IFN-γ (pg/ml) by ELISA in supernatants of PBMCs treated with either anti-EGFR antibody (anti-EGFR Ab) 5 μg/ml, anti-human HER2 antibody (anti-HER2 Ab) 5 μg/ml, CpG A ODN (CpG DNA) 5 μg/ml, anti-EGFR AB-CpG DNA 5 μg/ml, anti-HER2 Ab-CpG DNA 5 μg/ml, or left untreated (control). B) shows the quantification of Apo2L/TRAIL (pg/ml) by ELISA in supernatants of PBMCs treated with either anti-EGFR antibody (anti-EGFR Ab) 5 μg/ml, anti-human HER2 antibody (anti-HER2 Ab) 5 μg/ml, CpG A ODN (CpG DNA) 5 μg/ml, anti-EGFR AB-CpG DNA 5 μg/ml, anti-HER2 Ab-CpG DNA 5 μg/ml, or left untreated (control).

FIGS. 11A and 11B are graphs showing the inhibition of tumor growth and reduction of tumor volume in response to intratumoral or systemic administration of CpG DNA-conjugated anti-neu antibody to (neu-N)-transgenic mice. (A) untreated controls. (B) CpG DNA-conjugate anti-body treated cells.

FIG. 12 is a graph showing the inhibition of EGFR+ HT-29 tumor growth following administration of CpG DNA-conjugated anti-EGFR antibody.

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a nucleic acid” includes one or more nucleic acids, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

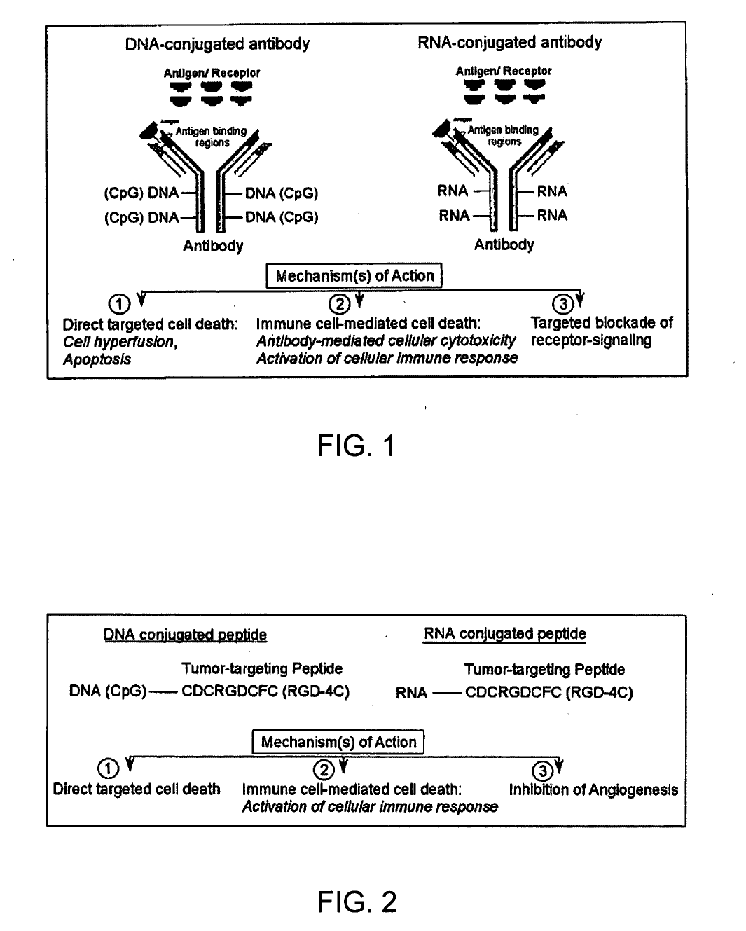

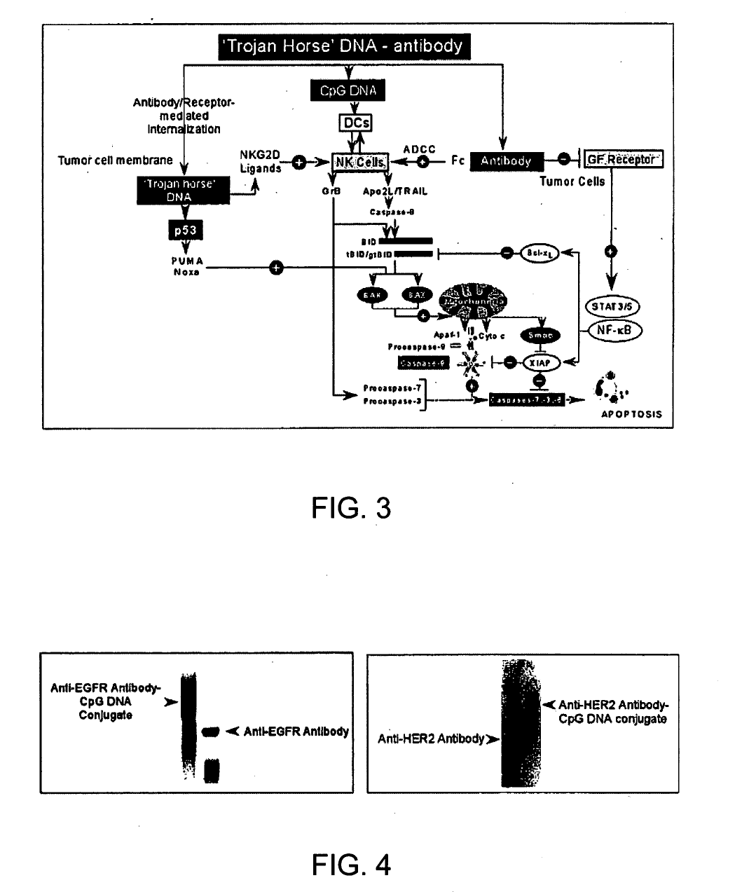

The introduction of immunostimulatory DNA-conjugated or RNA-conjugated antibodies/peptides activates death signaling in targeted cells (e.g., neoplastic cells) (FIG. 1 and FIG. 2). While not being bound by theory, and in contrast to the effects of genotoxic chemotherapeutic agents, use of DNA-conjugated or RNA-conjugated antibodies/peptides enables the activation of death signaling in targeted cells without corresponding effects on normal tissues that do not express the targeted molecule or express significantly lower levels of the molecule compared to neoplastic cells (FIG. 3).

In addition, immunostimulatory DNA-conjugated or RNA conjugated antibodies may simultaneously activate the immune system, recruit immune effector cells to the targeted cells, and sensitize tumor cells to immunologic cytotoxicity (e.g., by simultaneous blockade of growth factor-mediated signaling). The immune effector cells cooperate with direct DNA- or RNA-induced death signaling to induce apoptosis of tumor cells. Also, the tumor antigens released by apoptotic tumor cells, for example, are presented by dendritic cells (DCs) to generate long lasting adaptive antitumor immune responses. This approach may allow selective targeting and immunologic elimination of tumor cells without toxicity to normal cells, by activating intracellular death signaling in such cells.

Treatment of EGFR-expressing cancer cells with CpG DNA-conjugated anti-EGFR antibody or HER2/neu-expressing cancer cells with CpG DNA-conjugated anti-HER2/neu antibodies results in direct target receptor-specific death in the absence of PBMCs. The deregulated cell-cell fusion of targeted cells in response to treatment with nucleotide-conjugated antibodies results in the formation of coalesced (hybrid or multinucleated) cells with a limited lifespan and impaired replicating ability. This novel form of targeted cell death (cell hyperfusion) is not observed in response to treatment with unconjugated parent antibodies (anti-EGFR antibody or anti-HER2/neu antibodies) or free CpG DNA.

Cell hyperfusion may be observed by methods which assay for cell survival/proliferation including, but not limited to phase contrast microscopy, trypan blue exclusion, crystal violet staining, detection of coalesced cell bodies and/or detection of formation of multinucleate cell bodies.

In one aspect, DNA-conjugated or RNA-conjugated polypeptides/peptides simultaneously activate antitumor immune responses in the milieu of the tumor cells and inhibit of tumor angiogenesis. In a related aspect, polypeptides/peptides targeting the tumor cell, tumor vasculature, or tumor microenvironment aid in the delivery of immunostimulatory DNA/RNA to the tumor, and also inhibit tumor angiogenesis.

In one aspect, the conjugates of the present invention are used alone or in combination with other anticancer such as chemotherapeutic agents ionizing radiation, hormonal therapy, cytokines, immunotherapy, cellular therapy, vaccines, monoclonal antibodies, antiangiogenic agents, targeted therapeutics (small molecule drugs), or biological therapies. For example, chemotherapeutic agents include, but are not limited to, antitumor alkylating agents such as Mustards (mechlorethamine HCl, melphalan, chlorambucil, cyclophosphamide, ifosfamide, busulfan), Nitrosoureas (BCNU/carmustine, CCNU/lomustine, MeCCNU/semustine, fotemustine, streptozotocin), Tetrazines (dacarbazine, mitozolomide, temozolomide), Aziridines (thiotepa, mitomycin C, AZQ/diaziquone), procarbazine HCl, hexamethylmelamine, adozelesin; cisplatin and its analogues, cisplatin, carboplatin, oxaliplatin; antimetabolites, methotrexate, other antifolates, 5-fluoropyrimidines (5-fluorouracil/5-FU), cytarabine, azacitidine, gemcitabine, 6-thiopurines (6-mercaptopurine, thioguanine), hydroxyurea; topoisomerase interactive agents epipodophyllotoxins (etoposide, teniposide), camptothecin analogues (topotecan HCl, irinotecan, 9-aminocamptothecin), anthracyclines and related compounds (doxorubicin HCl, liposomal doxorubicin, daunorubicin HCl, daunorubicin HCl citrate liposomal, epirubicin, idarubicin), mitoxantrone, losoxantrone, actinomycin-D, amsacrine, pyrazoloacridine; antimicotubule agents Vinca alkaloids (vindesine, vincristine, vinblastine, vinorelbine), the taxanes(paclitaxel, docetaxel), estramustine; fludarabine, 2-chlorodeoxyadenosine, 2′-deoxycoformycin, homoharringtonine, suramin, bleomycin, L-asparaginase, floxuridine, capecitabine, cladribine, leucovorin, pentostatin, retinoids (all-trans retinoic acid, 13-cis-retinoic acid, 9-cis-retinoic acid, isotretinoin, tretinoin), pamidronate, thalidomide, cyclosporine; hormonal therapies antiestrogens (tamoxifen, toremifene, medroxyprogesterone acetate, megestrol acetate), aromatase inhibitors (aminoglutethimide, letrozole/femara, anastrozole/arimidex, exemestane/aromasin, vorozole), gonadotropin-releasing hormone analogues, antiandrogens (flutamide, casodex), fluoxymeterone, diethylstilbestrol, octreotide, leuprolide acetate, zoladex; steroidal and non-steroidal anti-inflammatory agents (dexamethasone, prednisone); Monoclonal antibodies including, but not limited to, anti-HER2/neu antibody (herceptin/trastuzumab), anti-EGFR antibody (cetuximab/erbitux, ABX-EGF/panitumumab, nimotuzumab), anti-CD20 antibody (rituxan/rituximab, ibritumomab/Zevalin, tositumomab/Bexxar), anti-CD33 antibody (gemtuzumab/Mylo Targ), alemtuzumab/Campath, bevacizumab/Avastin; and small molecule inhibitors.

As used herein “immune effector cells” include T cells, NK cells, B cells, macrophages, and dendritic cells (DC).

As used herein “a tumor targeting peptide” includes polymers containing fewer than 100 amino acids, where the polymer specifically binds to a cellular component of a tumor cell, tumor vasculature, and/or a component of a tumor microenvironment.

As used herein, “neoplasm,” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, melanoma, sarcoma, liver, kidney, lung, testicular, breast, ovarian, pancreatic, brain, head and neck, melanoma, leukemia, lymphoma cancer, and the like.

A used herein “subject,” including grammatical variations thereof, means a human or vertebrate animal including a dog, cat, horse, cow, pig, sheep, goat, chicken, monkey, rat, and mouse.

As used herein “conjugation,” including grammatical variations thereof, means directly linking, coupling, binding and the like of the foreign DNA with target-specific antibodies and/or peptides, either chemically, electrostatically, non-covalently, or by other techniques. For example, an isolated antibody-nucleic acid conjugate or peptide-nucleic acid as presently disclosed would fall under this definition.

An “immunostimulatory nucleic acid sequence” (INAS) refers to a nucleic acid molecule that is a pathogen-associated molecular pattern (PAMP) or other motif that can activate immune cells, including, but not limited to, CpG DNA (CpG), herpes simplex virus (HSV) DNA, double stranded RNA (dsRNA), and single stranded RNA (ssRNA). In a related aspect, the INAS may be a coding or non-coding sequence. For example, a CpG may be SEQ ID NO:1, in one illustrative example.

j In one aspect, such an immunostimulatory nucleic acid molecule is CpG (i.e., “CpG DNA” or DNA containing a cytosine followed by guanosine and linked by a phosphate bond) and bind to Toll-like receptors (TLRs) on immune effector cells (e.g., T cells, NK cells, B cells, and dendritic cells (DC)). In a related aspect, TLRs detect pathogens on the basis of motifs termed pathogen-associated molecular patterns (PAMPS) displayed on an invading organism.

In one embodiment, the invention provides an immunostimulatory nucleic acid sequence containing a CpG motif represented by the formula:
5′N₁X₁CGX₂N₂3′

where at least one nucleotide separates consecutive CpGs; X₁ is adenine, guanine, or thymine; X₂ is cytosine or thymine; N is any nucleotide and N₁+N₂ is from about 0-26 bases with the proviso that N₁ and N₂ do not contain a CCGG quadmer or more than one CCG or CGG trimer; and the nucleic acid sequence is from about 8-30 bases in length.

In another embodiment, the invention provides an isolated immunostimulatory nucleic acid sequence contains a CpG motif represented by the formula:
5′N₁X₁X₂CGX₃X₄N₂3′

where at least one nucleotide separates consecutive CpGs; X₁ X₂ include GpT, GpG, GpA, ApT and ApA; X₃ X₄ include TpT or CpT; N is any nucleotide and N₁+N₂ is from about 0-26 bases with the proviso that N₁ and N₂ do not contain a CCGG quadmer or more than one CCG or CGG trimer; and the nucleic acid sequence is from about 8-30 bases in length.

In a related aspect, the immunostimulatory nucleic acid sequences of the invention include X₁ X₂ selected from GpT, GpG, GpA and ApA and X₃ X₄ is selected from TpT, CpT and GpT. For facilitating uptake into cells, CpG containing immunostimulatory nucleic acid molecules may be in the range of 8 to 30 bases in length. However, nucleic acids of any size (even many kb long) are immunostimulatory if sufficient immunostimulatory motifs are present, since such larger nucleic acids are degraded into oligonucleotides inside of cells. In another aspect, synthetic oligonucleotides do not include a CGG quadmer or more than one CCG or CGG trimer at or near the 5′ and/or 3′ terminals and/or the consensus mitogenic CpG motif is not a palindrome. Prolonged immunostimulation can be obtained using stabilized oligonucleotides, where the oligonucleotide incorporates a phosphate backbone modification. For example, the modification is a phosphorothioate or phosphorodithioate modification. More particularly, the phosphate backbone modification occurs at the 5′ end of the nucleic acid for example, at the first two nucleotides of the 5′ end of the nucleic acid. Further, the phosphate backbone modification may occur at the 3′ end of the nucleic acid for example, at the last five nucleotides of the 3′ end of the nucleic acid.

In one aspect, the CpG DNA is in the range of between 8 to 30 bases in size when it is an oligonucleotide. Alternatively, CpG dinucleotides can be produced on a large scale in plasmids, which after being administered to a subject are degraded into oligonucleotides. In another aspect, nucleic acid molecules have a relatively high stimulation index with regard to B cell, monocyte and/or natural killer cell responses (e.g., cytokine, proliferative, lytic, or other responses).

Exemplary sequences include: 5′ gsgsGGACGACGTCGTGgsgsgsgsgsG 3′ (SEQ ID NO: 1) and 5′ gsgsGGGAGCATGCTGgsgsgsgsgsG 3′ (SEQ ID NO: 2).

A “stabilized nucleic acid molecule” shall mean a nucleic acid molecule that is relatively resistant to in vivo degradation (e.g., via an exo- or endo-nuclease). Stabilization can be a function of length or secondary structure. Unmethylated CpG containing nucleic acid molecules that are tens to hundreds of kbs long are relatively resistant to in vivo degradation. For shorter immunostimulatory nucleic acid molecules, secondary structure can stabilize and increase their effect. For example, if the 3′ end of a nucleic acid molecule has self-complementarily to an upstream region, so that it can fold back and form a sort of stem loop structure, then the nucleic acid molecule becomes stabilized and therefore exhibits more activity.

In one aspect, stabilized nucleic acid molecules of the instant invention have a modified backbone. For use in immune stimulation, stabilized nucleic acid molecules may include phosphorothioate (i.e., at least one of the phosphate oxygens of the nucleic acid molecules is replaced by sulfur) or phosphorodithioate modified nucleic acid molecules. More particularly, the phosphate backbone modification occurs at the 5′ end of the nucleic acid for example, at the first two nticleotides of the 5′ end of the nucleic acid. Further, the phosphate backbone modification may occur at the 3′ end of the nucleic acid for example, at the last five nucleotides of the 3′ end of the nucleic acid. In addition to stabilizing nucleic acid molecules, as reported further herein, phosphorothioate-modified nucleic acid molecules (including phosphorodithioate-modified) can increase the extent of immune stimulation of the nucleic acid molecule. For example, unmethylated CpG containing nucleic acid molecules having a phosphorothioate backbone have been found to activate B-cell activity, while unmethylated CpG containing nucleic acid molecules having a phosphodiester backbone have been found to activate monocytic (macrophages, dendritic cells and monocytes) and NK cells. Phosphorothioate CpG oligonucleotides with human motifs are also strong activators of monocytic and NK cells.

Other stabilized nucleic acid molecules include: nonionic DNA analogs, such as alkyl- and aryl-phosphonates (in which the charged phosphonate oxygen is replaced by an alkyl or aryl group), phosphodiester and alkylphosphotriesters, in which the charged oxygen moiety is alkylated. Nucleic acid molecules which contain a diol, such as tetraethylenglycol or hexaethyleneglycol, at either or both termini have also been shown to be substantially resistant to nuclease degradation. In one aspect, the nucleic acid molecules contain peptide bonds (i.e., peptide nucleic acids: PNAs).

For immunostimulatory nucleic acid molecules (INAS) of the present invention, the INAS may be coupled with a peptide or polypeptide in a number of ways including, but not limited to, conjugation (linkage). The polynucleotide portion can be coupled with the peptide or polypeptide portion of a conjugate involving covalent and/or non-covalent interactions. Generally, an INAS and peptide or polypeptide are linked in a manner that allows enhanced or facilitated uptake of the conjugate by a tumor or targeted cell.

The link between the peptide or polypeptide and INAS can be made at the 3′ or 5′ end of the INAS, or at a suitably modified base at an internal position in the INAS. If the peptide or polypeptide contains a suitable reactive group (e.g., an N-hydroxysuccinimide ester) it can be reacted directly with the N⁴ amino group of cytosine residues. Depending on the number and location of cytosine residues in the INAS, specific coupling at one or more residues can be achieved.

The polypeptide molecule of the conjugate can be an immunoglobulin. As used herein, the term “immunoglobulin” includes natural or artificial mono- or polyvalent antibodies including, but not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA, and IgY), class (e.g., IgG1, IgG2, IgG3, IgG4, IgA1, and IgA2) or subclass of immunoglobulin molecule.

Antibodies of the invention include antibody fragments that include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. In one aspect, the antibodies are human, murine (e.g., mouse and rat), donkey, sheep, rabbit, goat, guinea pig, camel, horse, or chicken. Further, such antibodies may be humanized versions of animal antibodies. The antibodies of the invention may be monospecific, bispecific, trispecific, or of greater multispecificity.

The antibodies of the invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Further, antibodies and antibody-like binding proteins may be made by phage display.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example; in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

In one embodiment, an antibody-nucleic acid conjugate is disclosed including an antibody that specifically binds to a cellular component of a tumor cell, tumor vasculature, and/or a component of a tumor microenvironment. A tumor microenvironment may contain epithelial cells, basement membrane, fibroblasts, stromal cells, and/or myofibroblasts, which surround the tumor. In a further related aspect, such cells surrounding the tumor may express functional CLIC4. Further, the conjugate has a binding affinity of at least 1 nM to 20 nM, including that such conjugate triggers cell hyperfusion between tumor cells in vitro subsequent to binding of the cellular component of the tumor cells.

In another aspect, the cellular components include, but are not limited to, epidermal growth factor receptor (EGFR, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family; platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family; TRK receptor family; ephrin (EPH) receptor family; AXL receptor family; leukocyte tyrosine kinase (LTK) receptor family; TIE receptor family, angiopoietin 1,2; receptor tyrosine kinase-like orphan receptor (ROR) receptor family; discoidin domain receptor (DDR) family; RET receptor family; KLG receptor family; RYK receptor family; MuSK receptor family; Transforming growth factor β (TGF-β) receptors, TGF-β; Cytokine receptors, Class I (hematopoietin family) and Class II (interferon/IL-10 family) receptors, tumor necrosis factor (TNF) receptor superfamily (TNFRSF), death receptor family; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), β-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/G[DP-L fucose: β-Dgalactose 2-α-Lfucosyltransferase (LDLR/FUT) fusion protein, HLA-A2. arginine to isoleucine exchange at residue 170 of the α-helix of the α2-domain in the HLA-A2 gene (HLA-A*201-R1701), HLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class I, NFYC, OGT, OS-9, pm1-RARalpha fusion protein, PRDX5, PTPRK, K-ras (KRAS2), N-ras (NRAS), HRAS, RBAF600, SIRT2, SNRPD1, SYT-SSX1 or —SSX2 fusion protein, Triosephosphate Isomerase, BAGE, BAGE-1, BAGE-2,3,4,5, GAGE-1,2,3,4,5,6,7,8, GnT-V (aberrant N-acetyl glucosaminyl transferase V, MGAT5), HERV-K-MEL, KK-LC, KM-HN-1, LAGE, LAGE-1, CTL-recognized antigen on melanoma (CAMEL), MAGE-A1 (MAGE-1), MAGE-A2, MAGE-A3, MAGE-A4, MAGE-A5, MAGE-A6, MAGE-A8, MAGE-A9, MAGE-A10, MAGE-A11, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gp100, gp100/Pmell7 (SILV), tyrosinase (TYR), TRP-1, HAGE, NA-88, NY-ESO-1, NY-ESO-1/LAGE-2, SAGE, Sp17, SSX-1,2,3,4, TRP2-INT2, carcino-embryonic antigen (CEA), Kallikrein 4, mammaglobin-A, OA1, prostate specific antigen (PSA), TRP-1/gp75, TRP-2, adipophilin, interferon inducible protein absent in melanoma 2 (AIM-2), BING-4, CPSF, cyclin D1, epithelial cell adhesion molecule (Ep-CAM), EphA3, fibroblast growth factor-5 (FGF-5), glycoprotein 250 (gp250), EGFR (ERBB1), HER-2/neu (ERBB2), interleukin 13 receptor α2 chain (IL13Ralpha2), IL-6 receptor, intestinal carboxyl esterase (iCE), alpha-feto protein (AFP), M-CSF, mdm-2, MUC1, p53 (TP53), PBF, PRAME, PSMA, RAGE-1, RNF43, RU2AS, SOX10, STEAP1, survivin (BIRC5), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA661, LDHC, MORC, SGY-1, SPO11, TPX1, NY-SAR-35, FTHL17, NXF2, TDRD1, TEX15, FATE, TPTE, immunoglobulin idiotypes, Bence-Jones protein, estrogen receptors (ER), androgen receptors (AR), CD40, CD30, CD20, CD19, CD33, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), β-human chorionic gonadotropin, squamous cell carcinoma antigen, neuron-specific enolase, heat shock protein gp96, GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr virus (EBV) proteins (EBV latent membrane proteins—LMP1, LMP2; others), Hepatitis B or C virus proteins, and HIV proteins.

In one embodiment, conjugates comprise peptides including, but not limited to, peptides that binds to a cellular component of a tumor cell, tumor vasculature, and/or a component of a tumor microenvironment. Conjugates may comprise peptides derived from phage display or other sources, including, but not limited to, αvβ1 integrin (CRRETAWAC (SEQ ID NO:5)), αvβ3 integrin (CDCRGDCFC (SEQ ID NO:6)/RGD-4C; RGDWXE (SEQ ID NO:7)), αvβ5 integrin (TRGDTF (SEQ ID NO:8)), αvβ6 (RGDLxxL (SEQ ID NO:9) or xxDLxxL (SEQ ID NO:10)), αIIβ3 (SRGDM (SEQ ID NO:11)), annexin V mimic for αvβ5 (VVISYSMPD (SEQ ID NO:12)), E-selectin (1ELLQAR (SEQ ID NO:13)), Endothelial cell mitochondria (CNGRC-GG-(KLAKLAK)2 (SEQ ID NO:14)), Ephrin-A2 and Ephrin-A4 (CVSNPRWKC (SEQ ID NO:15), CHVLWSTRC (SEQ ID NO:16)), Fibronectin (CWDDGWLC (SEQ ID NO:17)), ICAM-I or von Willebrand factor (CPCFLLGCC (SEQ ID NO:18)/LLG-4C), lamin-1 (DFKLFAVY (SEQ ID NO:19)), P-selectin (EWVDV (SEQ ID NO:20)), MMP-9:integrin complex (D/E)(D/E)(G/L)W (SEQ ID NO:21), MMP-9 and MMP-2 (gelatinases) (CTTHWGFTLC (SEQ ID NO:22)), Type I cadherin on endothelium (N-Ac-CHAVC-NH2), Fit-1 region of VEGF NxxEIExYxxWxxxxxY (SEQ ID NO:23), KDR region of VEGF (HTMYYHHYQHHL (SEQ ID NO:24), ATWLPPR(SEQ ID NO:25)), VEGF receptor (WHSDMEWWYLLG (SEQ ID NO:26), RRKRRR (SEQ ID NO:27), Aminopeptidase N/CD13 (NGR), NG2 proteolgycan (TAASGVRSMH (SEQ ID NO:28), LTLRWVGLMS (SEQ ID NO:29)), Adrenal gland derived peptide (LMLPRAD (SEQ-ID NO:30)), Adipose Tissue derived peptide (CKGGRAKDC SEQ ID NO:3 1)), Brain derived peptide (SR1), Brain endothelium derived peptide (CLSSRLDAC (SEQ ID NO:32)), Glioma cell derived peptide (VGLPEHTQ (SEQ ID NO:33)), Neuroblastoma derived peptide (VPWMEPAYQRFL (SEQ ID NO:34)), Bone Marrow derived peptide (GGG, GFS, LWS), Breast cancer (HER2/neu) derived peptide (LTVxPWx (SEQ ID NO:35), LTVxPWY (SEQ ID NO:36), HER2 Ab/Trastuzumab mimotope—LLGPYELWELSH (SEQ ID NO:37)), Colon derived peptide (RPMC (SEQ ID NO:38)), Intestine derived peptide (YSGKWGW (SEQ ID NO:39)), Head and Neck Squamous Cell Cancer derived peptide (TSPLNIHNGQKL (SEQ ID NO:40)), Lung vasculature derived peptide (CGFELETC(SEQ ID NO:41)), Coronary artery endothelia derived peptide (NSVRDL(G/S) (SEQ ID NO:42), NSVSSx(S/A) (SEQ ID NO:43)), Lymphatic Vessel derived peptide (CGNKRTRGC (SEQ ID NO:44)/Lyp-1), Multiple Organ derived peptide (GVL, EGRx (SEQ ID NO:45), xFG(G/V) (SEQ ID NO:46)), Pancreatic Islet derived peptide (CVSSNPRWKC (SEQ ID NO:47), CHVLWSTRC (SEQ ID NO:48)), Pancreas derived peptide (SWCEPGWCR (SEQ ID NO:49)), Prostate derived peptide (AGG, DPRATPGS (SEQ ID NO:50), SMSIARL (SEQ ID NO:5 1), CGRRAGGSC (SEQ ID NO:52), GVL), Retina derived peptide (RDV, CSCFRDVCC (SEQ ID NO:53)), Teratogen ligand derived peptide (TPKTSVT (SEQ ID NO:54)), and Uterus derived peptide (GLSGGRS (SEQ ID NO:55)).

In one aspect, an αᵥβ₃ peptide can have the sequence characteristics of either the natural ligand of αᵥβ₃ or αᵥβ₃ itself at the region involved in αᵥβ₃-ligand interaction. In one aspect, an αᵥβ₃ peptide contains the RGD tripeptide and corresponds in sequence to the natural ligand in the RGD-containing region.

In one aspect, RGD-containing peptides have a sequence corresponding to the amino acid residue sequence of the RGD-containing region of a natural ligand of αᵥβ₃ such as fibrinogen, vitronectin, von Willebrand factor, laminin, thrombospondin, and the like ligands. The sequence of these αᵥβ₃ ligands are well known. Thus, an αᵥβ₃ peptide can be derived from any of the natural ligands.

In another aspect, an αᵥβ₃ peptide preferentially inhibits αᵥβ₃ binding to its natural ligand(s) when compared to other integrins. The identification of αᵥβ₃ peptides having selectivity for αᵥβ₃ can readily be identified in a typical inhibition of binding assay, such as the ELISA assay.

A peptide of the present invention typically comprises no more than about 100 amino acid residues, preferably no more than about 60 residues, more preferably no more than about 30 residues. Peptides of the invention can be linear or cyclic.

It should be understood that a subject peptide need not be identical to the amino acid residue sequence of an αᵥβ₃ natural ligand. Exemplary sequences include: CDCRGDCFC (SEQ ID NO:3) and GGCDGRCG (SEQ ID NO:4).

A peptide of the invention includes any analog, fragment or chemical derivative of a peptide whose amino acid residue sequence is shown herein. Therefore, a present peptide can be subject to various changes, substitutions, insertions, and deletions where such changes provide for certain advantages in its use. In this regard, an αᵥβ₃ peptide of this invention corresponds to, rather than is identical to, the sequence of a recited peptide where one or more changes are made and it retains the ability to function as an αᵥβ₃ peptide in one or more of the assays.

The term “analog” includes any peptide having an amino acid residue sequence substantially identical to a sequence specifically shown herein in which one or more residues have been conservatively substituted with a functionally similar residue and which displays the αᵥβ₃ activity as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The term “fragment” refers to any subject polypeptide having an amino acid residue sequence shorter than that of a polypeptide whose amino acid residue sequence is disclosed herein.

A peptide of the present invention can be synthesized by any of the techniques that are known to those skilled in the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like. An excellent summary of the many techniques available can be found in Steward et al., “Solid Phase Peptide Synthesis”, W. H. Freeman Co., San Francisco, 1969; Bodanszky, et al., “Peptide Synthesis”, John Wiley & Sons, Second Edition, 1976; J. Meienhofer, “Hormonal Proteins and Peptides”, Vol. 2, p. 46, Academic Press (New York), 1983; Merrifield, Adv. Enzymol., 32:221-96, 1969; Fields et al., Int. J. Peptide Protein Res., 35:161-214, 1990; and U.S. Pat. No. 4,244,946 for solid phase peptide synthesis, and Schroder et al., “The Peptides”, Vol. 1, Academic Press (New York), 1965 for classical solution synthesis. Appropriate protective groups usable in such synthesis are described in the above texts and in J. F. W. McOmie, “Protective Groups in Organic Chemistry”, Plenum Press, New York, 1973.

The methods of the present invention can be generally employed to link an INAS to a variety of amino acid polymers, including peptides and antibodies.

Such methods include, but are not limited to, activation of a carboxylic acid moiety on a peptide or antibody by the addition of an activating agent. Activating agents include HATU (O-(7-azabenzotriazol-1-yl)-N,N,N′,N′-tetramethyluronium hexafluorophosphate); HBTU (O-benzotriazol-1-yl)-N,N,N′,N′-tetramethyluronium hexafluorophosphate); TBTU (2-(1H-benzotriazo-1-yl)-1-1,3,3-tetramethyluronium hexafluorophosphate); TFFH (N,N′,N″,N″ -tetramethyluronium 2-fluoro-hexafluorophosphate); BOP (benzotriazol-1-yloxytris(dimethylamino)phosphonium hexafluorophosphate); PyBOP (benzotriazole-1-yl-oxy-tris-pyrrolidino-phosphonium hexafluorophosphate); EEDQ (2-ethoxy-1-ethoxycarbonyl-1,2-dihydro-quinoline); DCC (dicyclohexylcarbodiimide); DIPCDI (diisopropylcarbodiimide); HOBt (1-hydroxybenzotriazole); N-hydroxysuccinimide; MSNT (1-(mesitylene-2-sulfonyl)-3-nitro-1H-1,2,4-triazole); aryl sulfonyl halides, e.g. triisopropylbenzenesulfonyl chloride. Preferred activating agents are carbodiimides. In one aspect, activating agents are 1-ethyl-3-(3-dimethylaminopropyl) carbodiimide hydrochloride (EDC) and/or 1-cyclohexyl-3-(2-morpholinoethyl) carbodiimide (CDC).

The activated carboxylic acid moiety as described above reacts with the nucleophilic moiety on the INAS, under conditions known to the skilled practitioner as sufficient to promote the reaction of the activated carboxylic acid moiety with the nucleophilic moiety. Under appropriate conditions, a relatively low pH is maintained, i.e., a pH less than about 6.5. Under traditional methods (i.e., at higher pH levels) it is believed that the activated carboxylic acid and/or the activating agent hydrolyze quickly, reducing the efficiency of the conjugation reaction.

The methods of the present invention can be used to prepare a variety of conjugates. In one aspect, conjugates of the present invention include, but are not limited to, DNA-antibody conjugates, DNA-peptide conjugates, RNA-antibody conjugates, and RNA-peptide conjugates.

Following the conjugation reaction, the conjugate can be isolated by-a variety of methods familiar to those skilled in the art. For example, the reaction mixture can be applied to a column chromatography system and separated by size-exclusion.

In one embodiment, a method of identifying a conjugate of the present invention which induces cell death, cell maturation, and/or NKG2D ligand dependent signaling is disclosed including, contacting one or more cells in vitro with a test conjugate containing an antibody that specifically binds to a cellular component of a tumor cell, tumor vasculature, and/or a component of a tumor microenvironment or an integrin derived peptide containing an RGD motif or a CDGRC motif, where the antibody or peptide is conjugated to a nucleic acid comprising one or more immunostimulatory nucleic acid sequences, and where one or more of the nucleic acid sequences comprise a pathogen-associated molecular pattern (PAMP) and determining induction of a marker or a phenotypic change in the one or more cells in the presence or absence of immune cells, where the determined induction or change in the presence of the test nucleic acid conjugate in one or more cells is indicative of cell death signaling, cell maturation, and/or NKG2D ligand dependent signaling.

For example, if contacting causes (a) cells to fuse in the absence of immune cells, where the cells are tumor cells, (b) tumor cells to lyse in a mixture of PBMC cells and tumor cells, and (c) the induction of expression of one or more markers, which include, but are not limited to, CD86, IFN-γ, and/or Apo2L/TRAIL, where the cells are PBMC or dendritic cells (DC), the test conjugate is associated with the induction of cell death signaling, cell maturation, and/or NKG2D ligand dependent signaling.

Induction of expressed markers may be accomplished by cell sorting. Further, cells are obtained from the bone marrow of a non-fetal animal, including, but not limited to, human cells. Fetal cells may also be used.

Cell sorting may be by any method known in the art to sort cells, including sorting by fluorescent activated cell sorting (FACS) and Magnetic bead cell sorting (MACS). To sort cells by MACS, one labels cells with magnetic beads and passes the cells through a paramagnetic separation column. The separation column is placed in a strong permanent magnet, thereby creating a magnetic field within the column. Cells that are magnetically labeled are trapped in the column; cells that are not pass through. One then elutes the trapped cells from the column.

The present invention also provides pharmaceutical compositions comprising at least one compound capable of treating a disorder in an amount effective therefor, and a pharmaceutically acceptable vehicle or diluent. The compositions of the present invention may contain other therapeutic agents as described, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

Pharmaceutical compositions employed as a component of invention articles of manufacture can be used in the form of a solid, a solution, an emulsion, a dispersion, a micelle, a liposome, and the like, where the resulting composition contains one or more of the compounds described above as an active ingredient, in admixture with an organic or inorganic carrier or excipient suitable for enteral or parenteral applications. Compounds employed for use as a component of invention articles of manufacture may be combined, for example, with the usual non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used.

Invention pharmaceutical compositions may be administered by any suitable means, for example, orally, such as in the form of tablets, capsules, granules or powders; sublingually; buccally; parenterally, such as by subcutaneous, intradermal, intravenous, intramuscular, or intracisternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present conjugates may also be administered liposomally. In one aspect, the composition may be administered systemically, intratumorally, or peritumorally.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

The subjects treated in the above methods, in which cells targeted for modulation is desired, are mammals, including, but not limited to, cows, sheep, goats, horses, dogs, cats, guinea pigs, rats or other bovine, ovine, equine, canine, feline, rodent or murine species, and preferably a human being, male or female.

The term “therapeutically effective amount” means the amount of the subject compound that will elicit the biological or medical response of a tissue, system, animal or human that is being sought by the researcher, veterinarian, medical doctor or other clinician.

The term “composition,” as used herein, is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of′ and or “administering a” compound should be understood to mean providing a compound of the invention to the individual in need of treatment.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules where the active ingredient is mixed with an inert solid diluent, for example, calcium carbonate, calcium phosphate or kaolin, or as soft gelatin capsules where the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles).

In the treatment of a subject where cells are targeted for modulation, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In one embodiment, an aliquot of blood is extracted from a mammalian subject, preferably a human, and the aliquot of blood is treated ex vivo with the conjugate of the present invention. The effect of the conjugate is to modulate the activity of immune effector cells in the blood which are contained in the aliquot. The modified aliquot is then re-introduced into the subject's body by any route suitable for vaccination.

In one aspect, a method is disclosed including removing immune cells from a subject, contacting the cells with the conjugate ex vivo, and reintroducing the cells into the subject.

In one aspect, the volume of the aliquot is up to about 400 ml, from about 0.1 to about 100 ml, from about 5 to about 15 ml, from about 8 to about 12 ml, or about 10 ml, along with an anticoagulant (e.g., 2 ml sodium citrate).

In one aspect, the subject undergoes a course of treatments, such individual treatments comprising removal of a blood aliquot, treatment thereof as described above and re-administration of the treated aliquot to the subject. A course of such treatments may comprise daily administration of treated blood aliquots for a number of consecutive days, or may comprise a first course of daily treatments for a designated period of time, followed by an interval and then one or more additional courses of daily treatments.

In a related aspect, the subject is given an initial course of treatments comprising the administration of 4 to 6 aliquots of treated blood. In another preferred embodiment, the subject is given an initial course of therapy comprising administration of from 2 to 4 aliquots of treated blood, with the administration of any pair of consecutive aliquots being either on consecutive days, or being separated by a rest period of from 1 to 21 days on which no aliquots are administered to the patient, the rest period separating one selected pair of consecutive aliquots being from about 3 to 15 days. In another related aspect, the dosage regimen of the initial course of treatments comprises a total of three aliquots, with the first and second aliquots being administered on consecutive days and a rest period of 11 days being provided between the administration of the second and third aliquots.

In a further related aspect, additional courses of treatments following the initial course of treatments. For example, subsequent courses of treatments are administered at least about three weeks after the end of the initial course of treatments. In one aspect, the subject receives a second course of treatment comprising the administration of one aliquot of treated blood every 30 days following the end of the initial course of treatments, for a period of 6 months.

It will be appreciated that the spacing between successive courses of treatments should be such that the positive effects of the treatment of the invention are maintained, and may be determined on the basis of the observed response of individual subjects.

The following examples are intended to illustrate but not limit the invention.

### EXAMPLES

### Example 1

### Generation of Conjugated Antibodies or Peptides

Anti-human EGFR antibody, Anti-human HER2 antibody, and Anti-rat neu antibody

```
CpG DNA [CpG Oligodeoxynucleotides (ODN)]
CpG A ODN, 21.92 μM; CpG C ODN, 18.34 μM
CpG A ODN: Sequence: 5′gsgsGGACGACGTCGTGgsgsgsgsgsG
3′(Phosphate) (SEQ ID NO: 1)
          Type = DNA-PS; Size = 21; Epsilon 1/(mMcm) = 208; MW (g/mole)
                        = 6842
CpG C ODN: Sequence: 5′gsgsGGGAGCATGCTGgsgsgsgsgsG 3′
(Phosphate) (SEQ ID NO: 2)
          Type = DNA-PS; Size = 20; Epsilon 1/(mMcm) = 197.6; MW (g/mole)
                        = 6553
Tumor-targeting peptide sequences:
CDCRGDCFC (RGD-4C peptide) (SEQ ID NO: 3);
GGCDGRCG (SEQ ID NO: 4)
(CDGRC peptide, SEQ ID NO: 5)
```

500 μl of antibody peptide solution was transferred into eppendorf tubes, to which 540 μl of 0.1M imidazole was added (i.e., 3M imidazole diluted in PBS to 0.1 M). 5 mg of 1-ethyl-3-[3-dimiethylaminopropyl]carbodiimide hydrochloride (EDC) was mixed with CpG DNA (ODN) in a separate tube, and immediately mixed with either antibody imidazole or peptide imidazole solution (Ab:ODN molar ratio=1:30.6).

The tubes were vortexed until the contents were dissolved, and the solution was briefly centrifuged. An additional 250 μl of 0.1 M imidazole was added subsequent to centrifugations, and the resulting solution was incubated at 50° C. for 2 hours.

The non-reacted EDC, its by-products, and imidazole was removed by CENTRICON® filtration (Millipore Corporation, Billerica, Mass.). The samples were then assayed by SDS-PAGE gels and mass spectrometry to determine conjugation of the nucleotide to the antibody and/or peptide. A protein assay was performed to quantify antibody or peptide concentration.

SDS-PAGE/immunoblotting demonstrated that the DNA conjugated monoclonal antibodies were in fact generated (FIG. 4).

### Example 2

### Inhibition of EGFR Activity by CDG DNA-conjugated Anti-EGFR Antibody

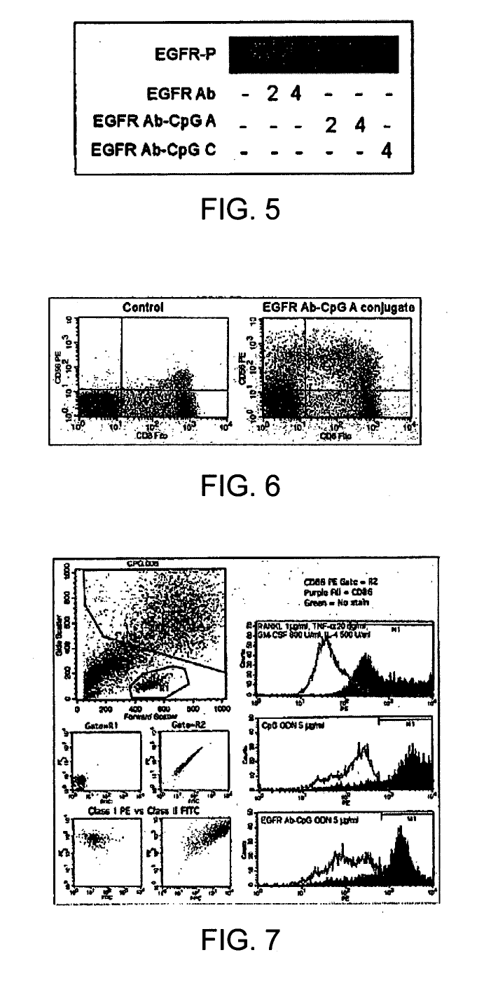

HT-29 colon carcinoma cells were cultured in 0.5% fetal bovine serum in the presence of either anti-EGFR antibody or CpG conjugated anti-EGFR antibody (Anti-EGFR Ab-CpG) and then stimulated with EGF (5 ng/ml) for 20 minutes at 37° C. Cells were then washed with ice-cold PBS containing 1 mM sodium orthovanadate, and cell lysates were subjected to Western blot analysis using antibodies that detect phospho-specific EGFR (tyrosine 1068; Cell Signaling). Treatment of HT-29 cells with anti-EGFR antibody or CpG DNA-conjugated antibody inhibited EGF-stimulated phosphorylation of EGFR (FIG. 5).

### Example 3

### Activation of Natural Killer Cells by CpG DNA-conjugated Anti-EGFR Antibody

Normal peripheral blood mononuclear cells (PBMCs)(Johns Hopkins leucopheresis Unit) were treated with either EGFR Ab-CpG DNA or EGFR Ab-Control DNA conjugated antibodies (4 μg/ml) for 3 d or left untreated. Cells were labeled with anti-CD56 phycoerythrin (CD56 PE) and anti-CD8 FITC (CD8 FITC) and then analyzed by flow cytometry. PBMCs showed increased numbers of CD56⁺ cells following stimulation with EGFR Ab-CpG conjugate, but not following treatment with EGFR Ab control DNA conjugate (FIG. 6).

### Example 4

### Maturation of Dendritic Cells by CpG DNA-conjugated Anti EGFR Antibody

Human monocytes were isolated from bone marrow mononuclear cells and cultured for 6 days in AIM5 media (with 10% human AB serum) and either of the following: (1) combination of the following cytokines: RANKL 1 μg/ml+TNF-α 20 ng/ml+GM-CSF 800 U/ml+IL-4 500 U/ml; (20 CpG oligonucleotide (CpG A ODN)(5 μg/ml)(without cytokines; (3) CpG ODN-conjugated anti-EGFR antibody (EGFR Ab-CpG ODN)(5 μg/ml)(without cytokines). Cells were harvested on day 7 and stained with antibodies to MHC class I PE, MHC class II FITC, and CD86-PE. Maturation of dendritic cells (DCs) was assessed by flow cytometric analysis of increased cell surface expression of the maturation marker CD86. CpG DNA-conjugated anti-EGFR antibody induced CD86 expression (i.e., maturation of DCs) that was similar to that observed in response to the cocktail of cytokines (FIG. 7).

### Example 5

### Effect of CpG DNA-conjugated Anti-EGFR Antibody on EGFR-expressing Tumor Cells

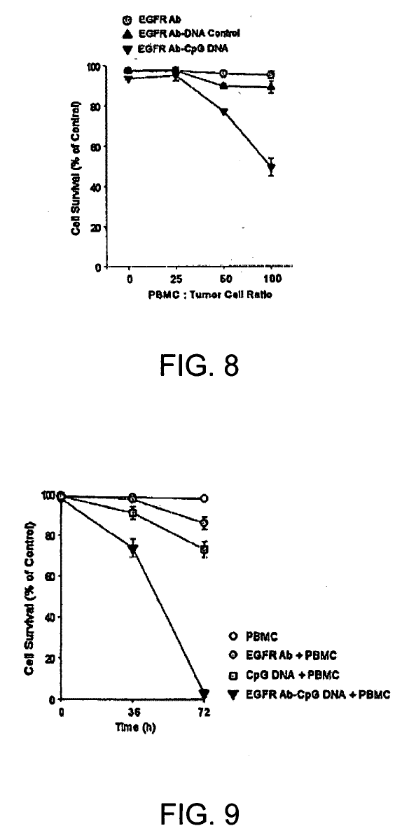

HT-29 colon carcinoma cells were labeled with ³H-thymidine (2.5 μCi/ml), trypsinized, washed with PBS, and treated with either EGFR-Ab, EGFR Ab-CpG DNA, or EGFR Ab-DNA control (4 μg/ml), were co-cultured in triplicate in 96-well plates (5×10³ cells/well) with PBMCs (pre-treated with either EGFR Ab-CpG DNA or EGFR Ab-DNA control conjugated antibodies or left untreated) at varying E:T ratios at 37° C. for 4 h. Cells were harvested onto a filter paper and cell death/survival was quantified by percent specific ³H-thymidine release. In contrast to treatment with EGFR-Ab (in the presence of untreated PBMCs) or EGFR Ab-DNA control (in the presence of EGFR Ab-DNA control treated PBMCs), treatment of HT-29 cells with EGFR Ab-CpG DNA (in the presence of EGFR Ab-CpG DNA-stimulated PBMCs) resulted in rapid death of HT-29 (FIG. 8).

HT-29 cells were cultured with either (1) EGFR-Ab (4 μg/ml)(with unstimulated PBMC); (2) EGFR Ab-CpG DNA (4 μg/ml)(with PBMCs pre-treated for 48 h with EGFR Ab-CpG DNA); or (3) PBMCs pre-treated for 48 h with CpG DNA (PBMC:tumor cell ratio=25). In contrast to treatment of HT-29 cells with either EGFR-Ab (in the presence of unstimulated PBMCs) or CpG DNA-stimulated PBMCs (in the absence of EGFR Ab), culture of HT-29 cells with EGFR Ab-CpG DNA (in the presence of EGFR Ab-CpG DNA-stimulated PBMCs) resulted in elimination of HT-29 cells over 72 h (FIG. 9).

### Example 6

### CpG DNA-Conjugated Antibodies [CpG DNA-Conjugated Anti-EGFR Antibody or CpG DNA-Conjugated Anti-HER2 Antibody] Induce Expression of Cytokines Interferon-γ (INF-γ) and Apo2L/TRIAL by Human Peripheral Blood Mononuclear Cells (PBMCs)

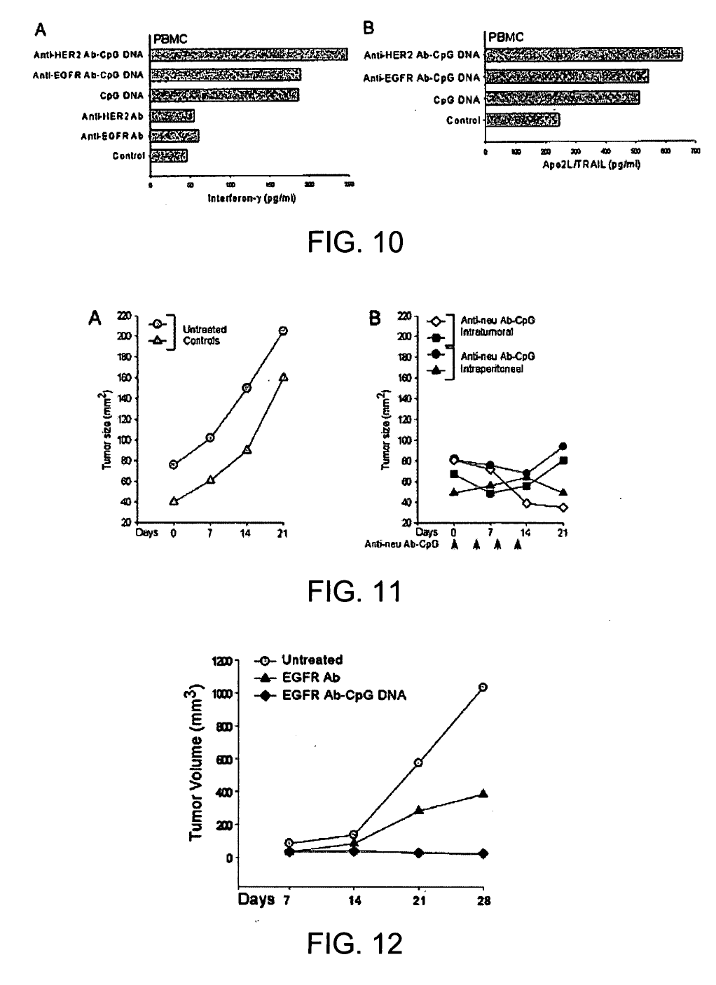

Human peripheral blood mononuclear cells (PBMCs) were treated with either anti-human EGFR antibody (anti-EGFR Ab) 5 μg/ml, anti-human HER2 antibody (anti-HER2 Ab) 5 μg/ml, CpG A ODN (CpG DNA) 5 μg/ml, nucleotide conjugated antibodies [anti-EGFR antibody-CpG DNA (anti-EGFR Ab-CpG DNA) or anti-HER2 antibody-CpG DNA (anti-HER2 Ab-CpG DNA) 5 μg/ml]. Levels of cytokines (INF-γ or Apo2L/TRAIL) in supernatants of PBMCs were assessed after 24 hours by ELISA (pg/ml). Treatment of PBMCs with either CpG DNA or CpG DNA conjugated antibodies increased expression of soluble INF-γ or Apo2L/TRAIL in cell supernatant (FIG. 10).

### Example 7

### DNA Conjugated Antibodies Induce a Novel Form of Targeted Cell Death—Cell Hyperfusion—that is Not Observed in Response to Any Known Class of Anticancer Agents

EGFR expressing human colon cancer cells (HT-29) were plated (5×10⁴ cells/ml) in the presence of either anti-EGFR antibody (anti-EGFR Ab) or CpG DNA-conjugated anti-EGFR antibody (anti-EGFR Ab-CpG)(5 μg/ml). Cells were followed by phase-contrast and time lapse microscopy for 96 h. Treatment with CpG DNA-conjugated Anti-EGFR antibody induced fusion of HT-29 cells and resulted in the formation of coalesced (hybrid or multinucleated) cells with a shorter lifespan and impaired replicating ability (hyperfusion) compared to cells that were treated with unconjugated anti-EGFR antibody.

EGFR expressing human breast cancer cells (MCF-7 or MDA-MB-468) were plated (5×10⁴ cells /ml) in the presence of either anti-EGFR antibody (anti-EGFR Ab) (2-8 μg/ml) or CpG DNA-conjugated anti-EGFR antibody (anti-EGFR Ab-CpG)(2-4 μg/ml). Treatment with CpG DNA-conjugated Anti-EGFR antibody induced hyperfusion of breast cancer cells and formed coalesced cell-bodies with a shorter lifespan and replicating ability compared to cells that were treated with the parental (unconjugated) anti-EGFR antibody.

HER2/neu-expressing human breast cancer cells (SKBr or MCF-7) were plated (5×10⁴ cells /ml) in the presence of either anti-human HER2/neu antibody (anti-HER2/neu Ab) or CpG DNA-conjugated anti-HER2/neu antibody (anti-HER2/neu Ab-CpG A DNA or anti-HER2/neu Ab-CpG C DNA)(5 μg/ml). Cell survival/proliferation was assessed by phase-contrast microscopy. Treatment with either CpG DNA-conjugated Anti-HER2/neu antibody induced hyperfusion of breast cancer cells and formed coalesced cell-bodies with a shorter lifespan and replicating abilities, which was not observed with cells treated by parental anti-HER2/neu antibody.

Mouse neu-expressing breast cancer cells (NT2 cells) were plated (5×10⁴ cells/ml) in the presence of either anti-neu antibody (anti-neu Ab) or CpG DNA conjugated anti-neu antibody (anti-neu Ab-CpG A DNA)(5 μg/ml). Cell survival/proliferation was assessed by phase-contrast microscopy and trypan-blue dye exclusion assays. Treatment with CpG DNA-conjugated anti-neu antibody induced hyperfusion of mouse neu-expressing breast cancer cells (NT2) and formed coalesced cell-bodies with reduced lifespan and replicating ability. Again, such hyperfusion and pronounced cell death was not induced by unconjugated antibody.

### Example 8

### CpG Conjugated Anti-neu Antibody Inhibits Growth of Spontaneous Tumors in HER2/neu Transgenic Mice

HER2/neu (neu/N)-transgenic mice bearing spontaneous mammary carcinomas were administered CpG DNA-conjugated anti-neu antibody (100 μg i.p. twice weekly for two weeks or 50 μg intratumoral twice weekly for two weeks), or left untreated. Analysis of tumor size and volume demonstrated marked inhibition of tumor growth and reduction of tumor volume following administration of CpG DNA-conjugated anti-neu antibody. (FIGS. 11A and 11B).

### Example 9

### CpG DNA Conjugated Anti-EGFR Antibody Inhibits Growth of Human EGFR⁺ Colon Cancer Xenografts in Nude Mice

BALB/c nude mice were injected subcutaneously with HT-29 human colon cancer cells (4×10⁶). Five days following tumor inoculation, mice were administered either anti-EGFR antibody or CpG DNA-conjugated anti-EGFR antibody (20 μg peri-tumoral twice weekly for three weeks), or left untreated. Analysis of tumor size and volume demonstrated marked inhibition of tumor growth following administration of CpG DNA-conjugated anti-EGFR antibody (FIG. 12). The inhibition of tumor growth in response to treatment with CpG DNA-conjugated anti-EGFR antibody was significantly greater than that of the unconjugated parent anti-EGFR antibody.

Although the invention has been described with reference to the above examples, it will be understood that modifications and variations are encompassed within the spirit and scope of the invention. Accordingly, the invention is limited only by the following claims.

What is claimed is:

1. An isolated antibody-nucleic acid or peptide-nucleic acid conjugate comprising:
i) an antibody or peptide that specifically binds to a cellular component of:
a) a tumor cell;
b) tumor vasculature; and/or
c) a component of a tumor microenvironment; and
ii) one or more immunostimulatory nucleic acid sequences (INAS), wherein one or more of the nucleic acid sequences comprise a pathogen-associated molecular pattern (PAMP).

2. The conjugate of claim 1, wherein the antibody is selected from the group consisting of a multispecific antibody, a human antibody, a humanized antibody, a chimeric antibody, a single chain antibody, an Fab fragment, an F(ab′) fragment, and an anti-idiotypic (anti-Id) antibody.

3. The conjugate of claim 1, wherein the cellular component is a tumor associated antigen or cell surface molecule.

4. The conjugate of claim 1, wherein the cellular component is selected from the group consisting of growth-factor receptors, co-stimulatory molecules, hormone receptors, cytokine receptors.

5. The conjugate of claim 1, wherein the cellular component is selected from the group consisting of epidermal growth factor receptor (EGFR, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family; platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family; TRK receptor family; ephrin (EPH) receptor family; AXL receptor family; leukocyte tyrosine kinase (LTK) receptor family; TIE receptor family, angiopoietin 1,2; receptor tyrosine kinase-like orphan receptor (ROR) receptor family; discoidin domain receptor (DDR) family; RET receptor family; KLG receptor family; RYK receptor family; MuSK receptor family; Transforming growth factor β (TGF-β) receptors, TGF-β; Cytokine receptors, Class I (hematopoietin family) and Class II (interferon/IL-10 family) receptors, tumor necrosis factor (TNF) receptor superfamily (TNFRSF), death receptor family; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), β-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: β-Dgalactose 2-α-Lfucosyltransferase (LDLR/FUT) fusion protein, HLA-A2. arginine to isoleucine exchange at residue 170 of the α-helix of the α2-domain in the HLA-A2 gene (HLA-A*201-R170I), HLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class I, NFYC, OGT, OS-9, pml-RARalpha fusion protein, PRDX5, PTPRK, K-ras (KRAS2), N-ras (NRAS), HRAS, RBAF600, SIRT2, SNRPD1, SYT-SSX1 or —SSX2 fusion protein, Triosephosphate Isomerase, BAGE, BAGE-1, BAGE-2,3,4,5, GAGE-1,2,3,4,5,6,7,8, GnT-V (aberrant N-acetyl glucosaminyl transferase V, MGAT5), HERV-K-MEL, KK-LC, KM-HN-1, LAGE, LAGE-1, CTL-recognized antigen on melanoma (CAMEL), MAGE-A1 (MAGE-1), MAGE-A2, MAGE-A3, MAGE-A4, MAGE-A5, MAGE-A6, MAGE-A8, MAGE-A9, MAGE-A10, MAGE-A11, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gp100, gp100/Pmel17 (SILV), tyrosinase (TYR), TRP-1, HAGE, NA-88, NY-ESO-1, NY-ESO-1/LAGE-2, SAGE, Sp17, SSX-1,2,3,4, TRP2-INT2, carcino-embryonic antigen (CEA), Kallikrein 4, mammaglobin-A, OA1, prostate specific antigen (PSA), TRP-1/gp75, TRP-2, adipophilin, interferon inducible protein absent in melanoma 2 (AIM-2), BING-4, CPSF, cyclin D1, epithelial cell adhesion molecule (Ep-CAM), EphA3, fibroblast growth factor-5 (FGF-5), glycoprotein 250 (gp250), EGFR (ERBB1), HER-2/neu (ERBB2), interleukin 13 receptor α2 chain (IL13Ralpha2), IL-6 receptor, intestinal carboxyl esterase (iCE), alpha-feto protein (AFP), M-CSF, mdm-2, MUC1, p53 (TP53), PBF, PRAME, PSMA, RAGE-1, RNF43, RU2AS, SOX10, STEAP1, survivin (BIRC5), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA661, LDHC, MORC, SGY-1, SPO11, TPX1, NY-SAR-35, FTHL17, NXF2, TDRD1, TEX15, FATE, TPTE, immunoglobulin idiotypes, Bence-Jones protein, estrogen receptors (ER), androgen receptors (AR), CD40, CD30, CD20, CD19, CD33, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), β-human chorionic gonadotropin, squamous cell carcinoma antigen, neuron-specific enolase, heat shock protein gp96, GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr virus (EBV) proteins (EBV latent membrane proteins—LMP1, LMP2; others), Hepatitis B or C virus proteins, and HIV proteins.

6. The conjugate of claim 5, wherein the cellular component is EGFR or HER2/neu.

7. The conjugate of claim 3, wherein the peptide is selected from the group consisting of αvβ1 integrin (CRRETAWAC (SEQ ID NO:5)), αvβ3 integrin (CDCRGDCFC (SEQ ID NO:6)/RGD-4C; RGDWXE (SEQ ID NO:7)), αvβ5 integrin (TRGDTF (SEQ ID NO:8)), αvβ6 (RGDLxxL (SEQ ID NO:9) or xxDLxxL (SEQ ID NO:10)), αIIβ3 (SRGDM (SEQ ID NO:11)), annexin V mimic for αvβ5 (VVISYSMPD (SEQ ID NO:12)), E-selectin (IELLQAR (SEQ ID NO:13)), Endothelial cell mitochondria (CNGRC-GG-(KLAKLAK)2 (SEQ ID NO:14)), Ephrin-A2 and Ephrin-A4 (CVSNPRWKC (SEQ ID NO:15), CHVLWSTRC (SEQ ID NO:16)), Fibronectin (CWDDGWLC (SEQ ID NO:17)), ICAM-I or von Willebrand factor (CPCFLLGCC (SEQ ID NO:18)/LLG-4C), lamin-1 (DFKLFAVY (SEQ ID NO:19)), P-selectin (EWVDV (SEQ ID NO:20)), MMP-9:integrin complex (D/E)(D/E)(G/L)W (SEQ ID NO:21), MMP-9 and MMP-2 (gelatinases) (CTTHWGFTLC (SEQ ID NO:22)), Type I cadherin on endothelium (N-Ac-CHAVC-NH2), Flt-1 region of VEGF NxxEIExYxxWxxxxxY(SEQ ID NO:23), KDR region of VEGF (HTMYYHHYQHHL (SEQ ID NO:24), ATWLPPR(SEQ ID NO:25)), VEGF receptor (WHSDMEWWYLLG (SEQ ID NO:26), RRKRRR (SEQ ID NO:27), Aminopeptidase N/CD13 (NGR), NG2 proteolgycan (TAASGVRSMH (SEQ ID NO:28), LTLRWVGLMS (SEQ ID NO:29)), Adrenal gland derived peptide (LMLPRAD (SEQ ID NO:30)), Adipose Tissue derived peptide (CKGGRAKDC SEQ ID NO:31)), Brain derived peptide (SR1), Brain endothelium derived peptide (CLSSRLDAC (SEQ ID NO:32)), Glioma cell derived peptide (VGLPEHTQ (SEQ ID NO:33)), Neuroblastoma derived peptide (VPWMEPAYQRFL (SEQ ID NO:34)), Bone Marrow derived peptide (GGG, GFS, LWS), Breast cancer (HER2/neu) derived peptide (LTVxPWx (SEQ ID NO:35), LTVxPWY (SEQ ID NO:36), HER2 Ab/Trastuzumab mimotope—LLGPYELWELSH (SEQ ID NO:37)), Colon derived peptide (RPMC (SEQ ID NO:38)), Intestine derived peptide (YSGKWGW (SEQ ID NO:39)), Head and Neck Squamous Cell Cancer derived peptide (TSPLNIHNGQKL (SEQ ID NO:40)), Lung vasculature derived peptide (CGFELETC(SEQ ID NO:41)), Coronary artery endothelia derived peptide (NSVRDL(G/S) (SEQ ID NO:42), NSVSSx(S/A) (SEQ ID NO:43)), Lymphatic Vessel derived peptide (CGNKRTRGC (SEQ ID NO:44)/Lyp-1), Multiple Organ derived peptide (GVL, EGRx (SEQ ID NO:45), xFG(GN) (SEQ ID NO:46)), Pancreatic Islet derived peptide (CVSSNPRWKC (SEQ ID NO:47), CHVLWSTRC (SEQ ID NO:48)), Pancreas derived peptide (SWCEPGWCR (SEQ ID NO:49)), Prostate derived peptide (AGG, DPRATPGS (SEQ ID NO:50), SMSIARL (SEQ ID NO:51), CGRRAGGSC (SEQ ID NO:52), GVL), Retina derived peptide (RDVI CSCFRDVCC (SEQ ID NO:53)), Teratogen ligand derived peptide (TPKTSVT (SEQ ID NO:54)), and Uterus derived peptide (GLSGGRS (SEQ ID NO:55)).

8. The conjugate of claim 1, wherein the INAS comprises a coding or non-coding nucleic acid.

9. The conjugate of claim 8, wherein the non-coding sequence is selected from the group consisting of double stranded DNA, single stranded DNA, CpG DNA (CpG), herpes simplex virus (HSV) DNA, double stranded RNA (dsRNA), CpG DNA (CpG), and single stranded RNA (ssRNA).

10. The conjugate of claim 9, wherein the non-coding sequence is CpG.

11. The conjugate of claim 10, wherein the CpG comprises SEQ ID NO:1.

12. The conjugate of claim 1, comprising:
i) an antibody that specifically binds to EGFR or HER/neu; and
ii) one or more immunostimulatory nucleic acid sequences, wherein
one or more of the nucleic acid sequences comprise a CpG DNA-sequence as set forth in SEQ ID NO:1.

13. The conjugate of claim 1, comprising:
i) a peptide containing an RGD motif or a CDGRC motif; and
ii) one or more immunostimulatory nucleic acid sequences, wherein
one or more of the nucleic acid sequences comprise a CpG DNA sequence as set forth in SEQ ID NO:1.

14. A method of treating a neoplastic disease comprising administering to a subject in need thereof, a composition comprising an antibody-nucleic acid conjugate or a peptide-nucleic acid conjugate, wherein the conjugate comprises:
i) an antibody or a peptide that specifically binds to a cellular component of:
a) a tumor cell,
b) tumor vasculature, and/or
c) a component of a tumor microenvironment; and
ii) one or more immunostimulatory nucleic acid sequences, wherein
one or more of the nucleic acid sequences comprise a pathogen-associated molecular pattern (PAMP).

15. The method of claim 14, wherein the conjugate is administered systemically, intratumorally, or peritumorally.

16. The method of claim 14, further comprising:
i) removing immune cells from the subject;
ii) contacting the cells of step (i) with the conjugate ex vivo; and
iii) reintroducing the cells of step (ii) into the subject.

17. The method of claim 14, further comprising administering an anticancer therapy selected from the group consisting of ionizing radiation, hormonal therapy, cytokines, immunotherapy, cellular therapy, vaccines, monoclonal antibodies, anti-angiogenic agents, and small molecule chemotherapeutic drugs.

18. The method of claim 14, wherein one or more of the nucleic acid sequences silences gene expression or induces intracellular death signaling.

19. The method of claim 18, wherein one or more of the nucleic acid sequences is double stranded RNA (dsRNA), short interfering RNA (siRNA), short hairpin RNA (shRNA), or micro RNA.

20. The method of claim 18, wherein at least one nucleic acid sequence encodes a peptide, a polypeptide, or a protein.

21. The method of claim 14, wherein the cellular component is selected from the group consisting of epidermal growth factor receptor (EGFR), HER2/neu, insulin-like growth factor receptor family, platelet derived growth factor receptor, interleukin-6 receptor, vascular endothelial growth factor (VEGF), VEGF receptor, CD40, CD28, estrogen receptors, cytokine receptors, CD20, CD19, CD33, MART-1/Melan-A, gp100, tyrosinase, TRP-1, MAGE-1, MAGE-3, MAGE-B1, MAGE-B2, BAGE, BAGE-1, GAGE-2, HAGE, LAGE, melanoma ubiquitous mutated 1, 2, 3 (MUM-1, 2, 3), β-catenin, carcino-embryonic antigen (CEA), HPV-E6, HPV-E7, mucin 1, prostate specific antigen (PSA), Epstein-Barr virus, alpha-feto protein (AFP), cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), β-human chorionic gonadotropin, squamous cell carcinoma antigen, Bence-Jones protein, neuron-specific enolase, heat shock protein gp96, heat shock protein 70-2 mutated (HSP70-2M), GM2, sargramostim, CTLA-4, prostatic acid phosphatase (PAP), NY-ESO-1, 707 alanine proline (707-AP), interferon inducible protein absent in melanoma 2 (AIM-2), adenocarcinoma antigen recognized by T cells 4 (ART-4), breakpoint cluster region-Abelson (Bcr-abl), CTL-recognized antigen on melanoma (CAMEL), carcinoembryogenic antigen peptide-1 (CAP-1), caspase-8 (CASP-8), cell division cycle 27 (CDC27), cyclin-dependent kinase 4 (CDK4), calcium-activated chloride channel-2 (CLCA2), cancer/testis antigen (CT), cyclophilin B (Cyp-B), elongation factor 2 (ELF2), epithelial cell adhesion molecule (Ep-CAM), ephrin type-A receptor 2, 3 (EphA2, 3), Ets variant gene 6/acute myeloid leukemia 1 gene ETS (ETC6-AML1), fibroblast growth factor 5 (FGF-5) fibronectin (FN), glycoprotein 250 (gp250), N-acetylglucosaminyltransferase V (GnT-V), arginine to isoleucine exchange at residue 170 of the α-helix of the α2-domain in the HLA-A2 gene (HLA-A*201-R170I), human signet ring tumor-2 (HST-2) human telomerase reverse transcriptase (hTERT), intestinal carboxyl esterase (iCE), interleukin 13 receptor α2 chain (IL-13Rα2), KIAA0205, low density lipid receptor/GDP-L fucose: β-Dgalactose 2-α-Lfucosyltransferase (LDLR/FUT), and Wilms' tumor gene (WT1).

22. The method of claim 14, wherein the conjugate comprises an antibody that specifically binds to EGFR or HER2/neu and one or more immunostimulatory nucleic acid sequences, wherein the one or more of the nucleic acid sequences comprise a CpG DNA sequence as set forth in SEQ ID NO:1.

23. The method of claim 14, wherein the peptide is selected from the group consisting of αvβ1 integrin (CRRETAWAC (SEQ ID NO:5)), αvβ3 integrin (CDCRGDCFC (SEQ ID NO:6)/RGD-4C; RGDWXE (SEQ ID NO:7)), αvβ5 integrin (TRGDTF (SEQ ID NO:8)), αvβ6 (RGDLxxL (SEQ ID NO:9) or xxDLxxL (SEQ ID NO:10)), αIIβ3 (SRGDM (SEQ ID NO:11)), annexin V mimic for αvβ5 (VVISYSMPD (SEQ ID NO:12)), E-selectin (IELLQAR (SEQ ID NO:13)), Endothelial cell mitochondria (CNGRC-GG-(KLAKLAK)2 (SEQ ID NO:14)), Ephrin-A2 and Ephrin-A4 (CVSNPRWKC (SEQ ID NO:15), CHVLWSTRC (SEQ ID NO:16)), Fibronectin (CWDDGWLC (SEQ ID NO:17)), ICAM-I or von Willebrand factor (CPCFLLGCC (SEQ ID NO:18)/LLG-4C), lamin-1 (DFKLFAVY (SEQ ID NO:19)), P-selectin (EWVDV (SEQ ID NO:20)), MMP-9:integrin complex (D/E)(D/E)(Q/L)W (SEQ ID NO:2 1), MMP-9 and MMP-2 (gelatinases) (CTTHWGFTLC (SEQ ID NO:22)), Type I cadherin on endothelium (N-Ac-CHAVC-NH2), Flt-1 region of VEGF NxxEIExYxxWxxxxxY(SEQ ID NO:23), KDR region of VEGF (HTMYYHHYQHHL (SEQ ID NO:24), ATWLPPR(SEQ ID NO:25)), VEGF receptor (WHSDMEWWYLLG (SEQ ID NO:26), RRKRRR (SEQ ID NO:27), Aminopeptidase N/CD13 (NGR), NG2 proteolgycan (TAASGVRSMH (SEQ ID NO:28), LTLRWVGLMS (SEQ ID NO:29)), Adrenal gland derived peptide (LMLPRAD (SEQ ID NO:30)), Adipose Tissue derived peptide (CKGGRAKDC SEQ ID NO:31)), Brain derived peptide (SR1), Brain endothelium derived peptide (CLSSRLDAC (SEQ ID NO:32)), Glioma cell derived peptide (VGLPEHTQ (SEQ ID NO:33)), Neuroblastoma derived peptide (VPWMEPAYQRFL (SEQ ID NO:34)), Bone Marrow derived peptide (GGG, GFS, LWS), Breast cancer (HER2/neu) derived peptide (LTVxPWx (SEQ ID NO:35), LTVxPWY (SEQ ID NO:36), HER2 Ab/Trastuzumab mimotope—LLGPYELWELSH (SEQ ID NO:37)), Colon derived peptide (RPMC (SEQ ID NO:38)), Intestine derived peptide (YSGKWGW (SEQ ID NO:39)), Head and Neck Squamous Cell Cancer derived peptide (TSPLNIHNGQKL (SEQ ID NO:40)), Lung vasculature derived peptide (CGFELETC(SEQ ID NO:41)), Coronary artery endothelia derived peptide (NSVRDL(G/S) (SEQ ID NO:42), NSVSSx(S/A) (SEQ ID NO:43)), Lymphatic Vessel derived peptide (CGNKRTRGC (SEQ ID NO:44)/Lyp-1), Multiple Organ derived peptide (GVL, EGRx (SEQ ID NO:45), xFG(GN) (SEQ ID NO:46)), Pancreatic Islet derived peptide (CVSSNPRWKC (SEQ ID NO:47), CHVLWSTRC (SEQ ID NO:48)), Pancreas derived peptide (SWCEPGWCR (SEQ ID NO:49)), Prostate derived peptide (AGG, DPRATPGS (SEQ ID NO:50), SMSIARL (SEQ ID NO:5 1), CGRRAGGSC (SEQ ID NO:52), GVL), Retina derived peptide (RDV, CSCFRDVCC (SEQ ID NO:53)), Teratogen ligand derived peptide (TPKTSVT (SEQ ID NO:54)), and Uterus derived peptide (GLSGGRS (SEQ ID NO:55)).

24. The method of claim 14, wherein the conjugate comprises a peptide containing an RGD motif or a CDGRC motif and one or more immunostimulatory nucleic acid sequences, wherein the one or more of the nucleic acid sequences comprise a CpG DNA sequence as set forth in SEQ ID NO:1.

25. The method of claim 14, wherein the neoplastic disease is cancer.

26. The method of claim 25, wherein the cancer is selected from the group consisting of head and neck cancers, aero-digestive cancers, gastro-intestinal cancers, esophageal cancers, stomach/gastric cancers, pancreatic cancers, hepato-biliary/liver cancers, colorectal cancers, anal cancers, small intestine cancers, genito-urinary cancers, urologic cancers, renal/kidney cancers, ureter cancers, testicular cancers, urethra/penis cancers, gynecologic cancers, ovarian/fallopian tube cancers, peritoneal cancers, uterine/endometrial cancers, cervical/vagina/vulva cancers, gestational trophoblastic disease, prostate cancers, bone cancers, sarcoma (soft tissue/bone), lung cancers, mesothelioma, mediastinum cancers, breast cancers, central nervous system cancers, brain cancers, melanoma, leukemia, lymphoma (Hodgkin's Disease and Non-Hodgkin's lymphoma), plasma cell neoplasms, myeloma, myelodysplastic syndrome, endocrine tumors, skin cancers, melanoma, thyroid cancers, parathyroid cancers, adrenal, pancreatic endocrine cancers, carcinoid, multiple endocrine neoplasia, AIDS-related malignancies, Cancer of unknown primary site, and various childhood cancers.

27. The method of claim 14, wherein the subject is a human.

28. A method of identifying a nucleic acid conjugate which induces cell death, cell maturation, and/or NKG2D ligand dependent signaling comprising:
i) contacting one or more cells in vitro with a test nucleic acid conjugate containing an antibody that specifically binds to a cellular component of a tumor cell, tumor vasculature, and/or a component of a tumor microenvironment or an integrin derived peptide containing an RGD motif or a CDGRC motif, wherein the antibody or peptide is conjugated toga nucleic acid comprising one or more immunostimulatory nucleic acid sequences, and wherein one or more of the nucleic acid sequences comprise a pathogen-associated molecular pattern (PAMP); and
ii) determining induction of a marker or a phenotypic change in the one or more cells of step (i) in the presence or absence of immune cells,
wherein the determined induction or change in the presence of the test nucleic acid conjugate in one or more cells of step (i) is indicative of cell death signaling, cell maturation, and/or NKG2D ligand dependent signaling.

29. The method of claim 28, wherein if the cells in step (i) are tumor cells, and in the absence immune cells, the contacting causes the cells to fuse, the test nucleic conjugate is associated with the induction of cell death signaling.

30. The method of claim 28, wherein if the cells in step (i) comprise a mixture of immune cells and tumor cells, and the contacting causes the tumor cells to lyse, the test nucleic conjugate is associated is associated with the induction of cell maturation and/or NKG2D ligand dependent signaling.

31. The method of claim 28, wherein if the cells in step (i) are PBMC cells or dendritic cells (DC), and the contacting causes the expression of one or more markers selected from the group consisting of CD86, IFN-γ, and/or Apo2L/TRAIL, the test nucleic-acid conjugate is associated with the induction of cell maturation.

32. A method of preventing or treating a neoplastic disease or infectious disease comprising administering to a subject in need thereof, a composition comprising an antibody-nucleic acid conjugate, wherein the conjugate comprises:
i) an antibody that specifically binds to a cellular component of an immune cell or a dendritic cell (DC);
ii) one or more immunostimulatory nucleic acid sequences (INAS), wherein
one or more of the nucleic acid sequences comprise a pathogen-associated molecular pattern (PAMP); and
iii) one or more tumor antigens or antigens from an infectious or pathogenic microorganism.

33. The method of claim 32, further comprising:
i) removing immune cells from the subject;
ii) contacting the cells of step (i) with the conjugate ex vivo; and
iii) reintroducing the cells of step (ii) into the subject.

34. The method of claim 32, further comprising administering an anticancer therapy selected from the group consisting of ionizing radiation, hormonal therapy, cytokines, immunotherapy, cellular therapy, vaccines, monoclonal antibodies, anti-angiogenic agents, and small molecule chemotherapeutic drugs.

35. The method of claim 32, wherein the infectious disease is caused by an infection selected from the group consisting of a microbial infection, fungal infection, parasitic infection, and viral infection.

36. The method of claim 32, wherein the subject is a human.
